SEQUENCE LISTING (13 sequences)

SEQ ID NO: 1 - protein, 40 aa, Artificial Sequence
  plectasin AMP
GFGCNGPWDEDDMQCHNHCKSIKGYKGGYCAKGGFVCKCY

SEQ ID NO: 2 - protein, 42 aa, Artificial Sequence
  eurocin AMP
GFGCPGDAYQCSEHCRALGGGRTGGYCAGPWYLGHPTCTCSF

SEQ ID NO: 3 - protein, 18 aa, Artificial Sequence
  linker peptide
GVHMVAGPGREPTGGGHM

SEQ ID NO: 4 - protein, 8 aa, Artificial Sequence
  AgrD1 bacterial pheromone
YSTCYFIM

SEQ ID NO: 5 - protein, 20 aa, Artificial Sequence
  multimerin1 guide peptide
MQKMTDQVNYQAMKLTLLQK

SEQ ID NO: 6 - protein, 49 aa, Artificial Sequence
  laterosporulin AMP
ACQCPDAISGWTHTDYQCHGLENKMYRHVYAICMNGTQVYCRTEWGSSC

SEQ ID NO: 7 - protein, 74 aa, Artificial Sequence
  MM1-laterosporulin gAMP
MQKMTDQVNYQAMKLTLLQKSGGGSACQCPDAISGWTHTDYQCHGLENKMYRHVYAICMNGTQVYCRTEWGSSC

SEQ ID NO: 8 - protein, 23 aa, Artificial Sequence
  alyteserin AMP
GLKDIFKAGLGSLVKGIAAHVAN

SEQ ID NO: 9 - protein, 48 aa, Artificial Sequence
  MM1-alyteserin gAMP
MQKMTDQVNYQAMKLTLLQKSGGGSGLKDIFKAGLGSLVKGIAAHVAN

SEQ ID NO: 10 - protein, 38 aa, Artificial Sequence
  cathelin-related anti-microbial peptide (CRAMP)
ISRLAGLLRKGGEKIGEKLKKIGQKIKNFFQKLVPQPE

SEQ ID NO: 11 - protein, 63 aa, Artificial Sequence
  MM1-CRAMP gAMP
MQKMTDQVNYQAMKLTLLQKSGGGSISRLAGLLRKGGEKIGEKLKKIGQKIKNFFQKLVPQPE

SEQ ID NO: 12 - DNA, 21 bp, Artificial Sequence
  VacA forward primer
atggaaatacaacaaacacac

SEQ ID NO: 13 - DNA, 19 bp, Artificial Sequence
  VacA reverse primer
ctgcttgaatgcgccaaac

(19) United States
(12) Patent Application Publication
(10) Pub. No.: US 2021/0052679 A1
(43) Pub. Date: Feb. 25, 2021

(54) PROBIOTIC DELIVERY OF GUIDED ANTIMICROBIAL PEPTIDES

(71) Applicant: Baylor University, Waco, TX (US)

(72) Inventors: Christopher Michel Kearney, Woodway, TX (US); Ankan Choudhury, Waco, TX (US); Patrick Ortiz, Waco, TX (US)

(73) Assignee: Baylor University, Waco, TX (US)

(21) Appl. No.: 16/997,036

(22) Filed: Aug. 19, 2020

Related U.S. Application Data
(60) Provisional application No. 62/888,757, filed on Aug. 19, 2019.

(51) Int. Cl. A61K 35/747; A61K 38/16; A61P 1/04; A61P 31/04; A23L 33/135; A23K 10/18

(52) U.S. Cl. CPC A61K 35/747; A61K 38/164; A61P 1/04; A23V 2002/00; A23L 33/135; A23K 10/18; A23Y 2240/41; A61P 31/04

(57) ABSTRACT

The present disclosure pertains to a treatment strategy to combat select bacteria in the gut, such as H. pylori. The strategy uses a probiotic-based system to express and deliver a guided antimicrobial peptide to the gut. The guided antimicrobial peptide is expressed from a hybrid gene that codes for an antimicrobial peptide fused to a guide peptide, the latter binding to a protein of the target bacterium. This technology can eliminate the target bacterium selectively and specifically from the gut microbiota. The specificity of the targeting, being at the strain, species or genus level, depends on the sequence of the guide peptide used to provide the targeting. The treatment can be administered orally, such as by using an ingestible probiotic.

### BACKGROUND

The present disclosure relates to a means of eliminating a specific gut bacterial species, such as Helicobacter pylori, without altering the microbiome.

The microbiota of the gut affects human health in many ways. The gut microbiome contains 100+ trillion bacteria and is largely involved in mediating the host's immune response while also performing other essential functions including the extraction of nutrients and energy from food. The bacterial makeup of the gut predisposes humans to health issues ranging from obesity to cancer to psychological disorders. Disruption to the microbiome (dysbiosis) results in an imbalance in the types and number of bacteria that comprise a person's normal, protective microflora. There are a number of factors that lead to dysbiosis including ingestion of pathogenic bacteria and antibiotic-mediated or immunosuppressive mediated depletion of the microbiome. Dysbiosis has been linked to numerous human diseases including both intestinal as well as extra-intestinal disorders. The literature indicates dysbiosis in the pathogenesis of IBS, inflammatory bowel disease, and colorectal cancer as well as allergies, cardiovascular disease, and mental illness. Additionally, gut microbiota have been implicated as precursor for autoimmune diseases given that severity and/or incidence of disease has been shown to be reduced in germ-free animal models.

In other cases, changes to gut bacteria result from ingestion of a dangerous pathogen that can produce an intestinal disease. There are few, if any, reported means to effectively knock out a specific bacterial species which is causing problems in the gut, either as an active pathogen or as a player in the microbiome that predisposes humans to various disorders.

Helicobacter pylori is a gut bacterium that is the primary cause of peptic ulcers and gastric cancer. Gastric cancer causes the third most fatalities worldwide among cancers and is especially common in the Far East (Bahkti et al., 2020). Only 1 in 5 patients survive gastric cancer 5 years after diagnosis. H. pylori is recognized by the International Agency for Research on Cancer as a Group 1 carcinogen. It is estimated that 4.4 billion people are infected with H. pylori, with developing countries having the highest infection rates (70% prevalence in Africa) (Hooi et al., 2017). In the United States, H. pylori occurs twice as frequently in the non-white population as in the white population (Everhart et al., 2000) and is associated with lower socio-economic status worldwide.

No commercial vaccine exists against H. pylori. Though some progress has been seen in lowered H. pylori prevalence in some countries using antibiotic treatment, large increases in antibiotic resistance rates are now being seen in H. pylori isolates. The prevalence of clarithromycin-resistance in H. pylori rose from 11% to 60% in just 4 years (2005-2009) in Korea, with similar increases recorded in China and Japan (Thung et al., 2016). Though the standard treatment is in fact a triple antibiotic therapy, antibiotic resistance rates continue to rise. Thus, it is difficult to see a path forward with H. pylori treatment via antibiotics. Other bacteria offer similar challenges.

### SUMMARY

The present disclosure pertains to a treatment strategy to combat select bacteria in the gut, such as H. pylori. The strategy uses a probiotic-based system for the expression and delivery of a guided antimicrobial peptide to the gut. The guided antimicrobial peptide is expressed from a hybrid gene in the probiotic bacterium's DNA, and can be the sequence coding for an antimicrobial peptide fused to the sequence coding for a guide peptide, with the latter peptide responsible for binding to a protein of the target bacterium. The fusing can occur with or without a linker sequence, that is, independent of the presence of a linker sequence. This technology can eliminate the target bacterium selectively and specifically from the gut microbiota. The specificity of the targeting, being at the strain, species or genus level, depends on the guide protein used to provide the targeting. The treatment can be administered orally, such as by using an ingestible probiotic.

Preferred embodiments described herein relate to a method for the control of a target bacterium such as H. pylori which does not involve antibiotics. For delivery of the active protein, this method uses engineered probiotic bacteria. Preferred embodiments utilize lactic acid bacteria, including Lactococcus and Lactobacillus species, such as Lactococcus lactis and Lactobacillus acidophilus, which are food grade bacterium that are safe for human consumption or have been granted GRAS status (Generally Regarded As Safe) by the FDA and are in widespread commercial use for processing dairy food products. Probiotics constitute a well-established technology which is inexpensive, highly scalable, and very successful commercially. These commercial traits make this technology especially amenable to large-scale application, particularly in developing countries.

The probiotic bacterium can be formulated as a recognizable food product that is commonly found in the probiotics market, such as dried yoghurt pellets, which can be stored without refrigeration for months. In this format, the product may be taken by travelers to foreign countries or by long-term expatriates or soldiers with food, perhaps twice per week, as a preventative (“prophylactic”) to disease. The treatment could also serve as a therapy, being eaten after the patient is sick.

The present technology is important and advantageous because it utilizes guided antimicrobial peptides that eliminate only the target bacterium while leaving all the other members of the microbial community undisturbed. The use of probiotic bacteria that are ingested and remain active in the digestive system in order to secrete the guided recombinant antimicrobial peptide directly in the gut of the patient is also significantly different from previous technologies.

### BRIEF DESCRIPTION OF DRAWINGS

FIG. 1 shows the pE-SUMOstar vector carrying AMP for expression in E. coli BL21 cells. SUMO protease site is between SUMO and A12C-AMP.

FIG. 2 shows expression of SUMO/AMP in E. coli and cleavage of AMP free of SUMO fusion partner.

FIG. 3 shows log values for minimum inhibitory concentrations (MIC) in M for non-targeted and targeted analogues of eurocin and plectasin against Bacillus subtilis, Enterococcus faecalis, Staphylococcus aureus and Staphylococcus epidermidis.

FIG. 4 shows the cell-kinetic profile for B. subtilis, S. epidermidis, S. aureus and E. faecalis (clockwise), created by plotting log CFU/ml of the bacteria grown in the presence of each peptide.

FIG. 5 shows biofilm inhibition activity evaluated by plotting the absorbance of crystal violet (540 nm) against the concentration of 4 AMPs on the 4 bacteria—B. subtilis, S. epidermidis, S. aureus and E. faecalis.

FIG. 6 shows results of a PCR analysis of stomach reverse gavage extracts demonstrating the presence of Lactococcus lactis harboring the empty vector, the vector with antimicrobial peptide, and the the vector with antimicrobial peptide with the guide peptide from multimerin in the stomachs of mice three days after ingestion.

FIG. 7 shows a vector for transformation of Lactococcus lactis in accordance with preferred embodiments described herein.

FIG. 8 shows the viability of E. coli in the presence of different antibiotic dilutions and supernatants of broth cultures of Lactococcus lactis secreting antimicrobial peptide with or without a guide peptide.

FIG. 9 shows an exemplary vector for Lactococcus lactis secretion of AMPs and gAMPs.

FIG. 10 shows results of qPCR on VacA gene, showing elimination of H. pylori by co-culturing in vitro with L. lactis expressing gAMPs or AMPs.

FIG. 11 shows growth of Lactobacillus plantarum after 24 hours co-culturing with L. lactis expressing empty vector (pTKR), AMPs (alyteserin, laterosporulin, or CRAMP), or gAMPs (MM1-alyteserin, MM1-laterosporulin, or MM1-CRAMP).

FIG. 12 shows growth of Escherichia coli after 24 hours co-culturing with L. lactis expressing empty vector (pTKR), AMPs (alyteserin, laterosporulin, or CRAMP), or gAMPs (MM1-alyteserin, MM1-laterosporulin, or MM1-CRAMP).

FIG. 13 shows a standard curve for CFU/μl of H. pylori culture with qPCR C_{T} values.

FIG. 14 shows a therapeutic test, with the CFU/μl of H. pylori vs days after inoculation, in mice treated with Lactococcus lactis probiotic secreting AMPs or gAMPs on Day 5 after inoculation with H. pylori.

FIG. 15 shows a prophylactic test, with the CFU/μl of H. pylori in mouse stomach fluid for control mice (Null) and mice inoculated with empty vector (pTKR) or Lactococcus lactis probiotic secreting AMPs or gAMPs (where MM1=Multimerin1 guide peptide), before inoculation with H. pylori on Day 4.

FIG. 16 shows the differences in taxonomic diversity for mouse stomach bacterial populations with four different treatments without the presence of H pylori: Antibiotic treatment, L. lactis probiotic with empty vector, buffer mock inoculation, probiotic expressing AMP, probiotic expressing gAMP.

FIG. 17 shows differences in relative abundance of four bacterial indicator species under different treatments; Staphylococcus and Acinetobacter are associated with dysbiosis while Lactobacillus and Muribacter are associated with microbiota health; Day 0 is before any treatment; Day 5 is after 5 days of H. pylori infection; Days 8 and 10 are 3 and 5 days, respectively, after various therapeutic treatments (probiotics with either empty vector or expressing AMP or gAMP).

FIG. 18 shows taxonomic differences (distance) in sequencing data for bacterial species found in mouse stomach in four treatment groups, Empty (probiotic carrying only an empty vector), Null (mock inoculation with buffer), Targeted (probiotic expressing gAMP), and Non-targeted (probiotic expressing AMP), compared to Empty.

FIG. 19 shows taxonomic differences (distance) in sequencing data for bacterial species found in mouse stomach in four treatment groups, Empty (probiotic carrying only an empty vector), Null (mock inoculation with buffer), Targeted (probiotic expressing gAMP), and Non-targeted (probiotic expressing AMP), compared to Null.

FIG. 20 shows cumulative taxonomic differences (Shannon entropy) accruing over five days in sequencing data for bacterial species found in mouse stomach after four different treatments: Empty (probiotic carrying only an empty vector), Null (mock inoculation with buffer), Targeted (probiotic expressing gAMP), and Non-targeted (probiotic expressing AMP).

### DETAILED DESCRIPTION OF PREFERRED EMBODIMENTS

The present disclosure relates to a means for targeting and eliminating a target bacterium using a probiotic that expresses and secretes a protein that kills the disruptive bacterium without harming other bacteria.

In preferred embodiments, the present technology pertains to a probiotic bacterium that has been transformed to include a DNA construct for a guided antimicrobial peptide. In preferred embodiments, the probiotic bacterium is a bacterium that is safe for human consumption, such as Lactococcus lactis. The sequence coding for the guided antimicrobial peptide includes the sequence coding for a targeting (guide) peptide fused to the sequence coding for an antimicrobial peptide and expressed by the probiotic bacterium as a hybrid protein. The guide peptide is specific for the target bacterium and limits the action of the antimicrobial peptide to that particular bacterium.

Accordingly, preferred embodiments described herein relate to a probiotic for the prevention or treatment of a condition caused by a target bacterium living in the gastrointestinal tract of a subject, comprising a probiotic bacterium. The probiotic bacterium is preferably a lactic acid bacterium, such as a Lactococcus bacterium, and preferably Lactococcus lactis. The probiotic bacterium has been transformed to comprise a DNA construct expressing a guided antimicrobial peptide, wherein the sequence coding for the guided antimicrobial peptide comprises the sequence coding for an antimicrobial peptide fused to the sequence coding for a guide peptide that binds to a protein of the target bacterium. The protein of the target bacterium may be a virulence factor. In preferred embodiments, the target bacterium is H. pylori and the virulence factor is VacA. The guide peptide may be multimerin-1. The guided antimicrobial peptide kills the target bacterium in the gastrointestinal tract of the subject. The guided antimicrobial peptide also minimally disrupts other bacteria found in the gastrointestinal tract of the subject when compared to unguided antimicrobial peptides, antibiotics, or other broad spectrum treatments.

As used herein, “minimally disrupts” means the guided antimicrobial peptide does not cause a disruption that would cause a health effect, as opposed to a technical change in bacterial abundance. “Minimally disrupts” also means the guided antimicrobial peptide does not significantly disrupt other non-target bacteria, where the disruption would cause a health effect.

Preferred embodiments relate to a probiotic system which delivers antimicrobial peptides (AMPs) to the gut. Antimicrobial peptides are natural products produced by plants, animals and fungi to protect against bacterial infection (Ngyuen et al., 2011). However, an AMP by itself has broad spectrum activity, similar to an antibiotic. The broad activity of antibiotics has been well-documented to lead to microbiota dysbiosis. Many publications have demonstrated connections between antibiotic-induced dysbiosis and rheumatoid arthritis, inflammatory bowel disease, diabetes, obesity and other disorders (for a review, see Keeney et al., 2014). This is one of the consequences of the overuse of antibiotics and nonselective AMPs share the same weakness. Exemplary AMPs used in preferred embodiments described herein include laterosporulin, alyteserin, and cathelin-related anti-microbial peptide (CRAMP).

To solve this problem of dysbiosis, the preferred embodiments described herein include a guide peptide fused to an AMP, produced from a corresponding guide-AMP hybrid gene of the probiotic bacterium. This enables the resulting guided AMP (gAMP) to bind specifically to the targeted bacterium such as H. pylori, leaving the commensal bacteria of the gut largely undisturbed. In this way, a probiotic expressing gAMP will multiply in the stomach and selectively kill the target pathogen, H. pylori without the health issues associated with antibiotics and other broad-spectrum treatments. Other targeted bacterium can be treated similarly, and H. pylori is used herein as an example.

In preferred embodiments, the specificity of the guide peptide described herein is based on the natural specificity of a bacterial virulence factor and the host receptor to which it binds. VacA is a virulence factor protein produced by all isolates of H. pylori (Fitchen et al., 2005). It is secreted but also adheres to the surface of H. pylori cells (Fitchen et al., 2005). VacA naturally binds to the human receptor protein, multimerin-1. Preferred embodiments described herein utilize the VacA-binding sequence (aa 321-340) of the multimerin-1 protein (Satoh et al., 2013) to serve as the guide peptide for the gAMPs. In this way, these gAMPs will be localized to the surface of the H. pylori via binding to VacA and the AMP portion can then act to destabilize the bacterial membrane and specifically kill the H. pylori cell.

The probiotic gAMPs described in preferred embodiments are distinct from similar technologies. They possess a selectivity not found in antibiotics and unguided AMPs. The use of probiotics makes it possible to produce probiotic gAMPs much more cheaply than gAMP proteins purified from a heterologous expression system or synthesized chemically. This combination of selectivity and low-cost scalability is essential for any replacement for cheap and abundant antibiotics to be successful commercially and therefore reach the intended patients.

Preferred embodiments disclosed herein relate to an edible Lactococcus lactis probiotic bacterium, wherein the probiotic bacterium has been transformed to comprise a DNA construct expressing a guided antimicrobial peptide, wherein the sequence coding for the guided antimicrobial peptide comprises the sequence coding for an antimicrobial peptide fused to the sequence coding for a guide peptide that binds to the VacA peptide of H. pylori, produced from the corresponding hybrid gene of the L. lactis bacterium, wherein the antimicrobial peptide is laterosporulin, alyteserin, or cathelin-related anti-microbial peptide, and wherein the guided antimicrobial peptide kills H. pylori in the gastrointestinal tract of the patient without causing a significant disruptive effect on other bacterial species. In other words, the probiotic bacterium expressing the guided antimicrobial peptide will not disrupt the taxonomic balance of the stomach microbiota and will not cause long-term damage.

Additional preferred embodiments relate to a method for treating a disease or condition associated with H. pylori by administering an edible probiotic to a subject, where the edible probiotic is ingested and remains active in the subject's gut long enough to secrete a guided antimicrobial peptide that kills H. pylori.

In another aspect of the present invention there is provided a probiotic composition including a therapeutically effective amount of a transformed probiotic L. lactis bacterium expressing a guided antimicrobial peptide and an acceptable excipient, adjuvant, carrier, buffer or stabiliser. A “therapeutically effective amount” is to be understood as an amount of an exemplary probiotic that is sufficient to show inhibitory effects on H. pylori. The actual amount, rate and time-course of administration will depend on the nature and severity of the condition or disease being treated. Prescription of treatment is within the responsibility of general practitioners and other medical doctors. The acceptable excipient, adjuvant, carrier, buffer or stabiliser should be non-toxic and should not interfere with the efficacy of the secreted antimicrobial protein. The precise nature of the carrier or other material will depend on the route of administration, which is preferably oral.

The L. lactis bacteria useful in the disclosed probiotic composition may be provided as a live culture, as a dormant material or a combination thereof. Those skilled in the art will appreciate that the L. lactis bacteria may be rendered dormant by, for example, a lyophilization process, as is well known to those skilled in the art.

An example of an appropriate lyophilization process may begin with a media carrying appropriate L. lactis bacteria to which an appropriate protectant may be added for cell protection prior to lyophilization. Examples of appropriate protectants include, but are not limited to, distilled water, polyethylene glycol, sucrose, trehalose, skim milk, xylose, hemicellulose, pectin, amylose, amylopectin, xylan, arabinogalactan, starch (e.g., potato starch or rice starch) and polyvinylpyrrolidone. Gasses useful for the lyophilization process include but are not limited to nitrogen and carbon dioxide.

In one aspect, the L. lactis bacteria in the disclosed probiotic composition may be provided as a dispersion in a solution or media. In another aspect, the L. lactis bacteria in the disclosed probiotic may be provided as a semi-solid or cake. In another aspect, the L. lactis bacteria in the disclosed probiotic may be provided in powdered form.

Quantities of appropriate L. lactis bacteria may be generated using a fermentation process. For example, a sterile, anaerobic fermentor may be charged with media, such as glucose, polysaccharides, oligosaccharides, mono- and disaccharides, yeast extract, protein/nitrogen sources, macronutrients and trace nutrients (vitamins and minerals), and cultures of the desired L. lactis bacteria may be added to the media. During fermentation, concentration (colony forming units per gram), purity, safety and lack of contaminants may be monitored to ensure a quality end result. After fermentation, the L. lactis bacteria cells may be separated from the media using various well known techniques, such as filtering, centrifuging and the like. The separated cells may be dried by, for example, lyophilization, spray drying, heat drying or combinations thereof, with protective solutions/media added as needed.

The probiotic compositions may be prepared in various forms, such as capsules, suppositories, tablets, food/drink and the like. The probiotic compositions may include various pharmaceutically acceptable excipients, such as microcrystalline cellulose, mannitol, glucose, defatted milk powder, polyvinylpyrrolidone, starch and combinations thereof.

The probiotic composition may be prepared as a capsule. The capsule (i.e., the carrier) may be a hollow, generally cylindrical capsule formed from various substances, such as gelatin, cellulose, carbohydrate or the like. The capsule may receive the probiotic bacteria therein. Optionally, and in addition to the appropriate probiotic bacteria, the capsule may include but is not limited to coloring, flavoring, rice or other starch, glycerin, caramel color and/or titanium dioxide.

The probiotic composition may be prepared as a suppository. The suppository may include but is not limited to the appropriate probiotic bacteria and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

The probiotic composition may be prepared as a tablet. The tablet may include the appropriate probiotic bacteria and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets. A capsule may also be used to contain the composition.

The probiotic composition may be formed as food or drink or, alternatively, as an additive to food or drink, wherein an appropriate quantity of probiotic bacteria is added to the food or drink to render the food or drink the carrier.

The concentration of probiotic bacteria in the probiotic composition may vary depending upon the desired result, the type of bacteria used, the form and method of administration, among other things. For example, a probiotic composition may be prepared having a count of probiotic bacteria in the preparation of no less than about 1×10⁶ colony forming units (CFUs) per gram, based upon the total weight of the preparation.

When lactic acid bacteria are used as gut expression vehicles, various dairy products, such as youghurt, youghurt pellets, or other milk products may be used as the physical carrier for oral administration, with or without the above mentioned adjuvants or carriers.

In another aspect, there is provided the use in the manufacture of a medicament of a therapeutically effective amount of a probiotic as defined above for administration to a subject.

The term “therapeutically effective amount” means a nontoxic but sufficient amount of the probiotic to provide the desired therapeutic effect. The amount that is “effective” will vary from subject to subject, depending on the age and general condition of the individual, the particular concentration and composition being administered, and the like. Thus, it is not always possible to specify an exact effective amount. However, an appropriate effective amount in any individual case may be determined by one of ordinary skill in the art using routine experimentation. Furthermore, the effective amount is the concentration that is within a range sufficient to permit ready application of the formulation so as to deliver an amount of the drug that is within a therapeutically effective range.

The probiotic in its final form is expected to have a very low production cost and be highly scalable. In addition, it should have a long shelf life and not require refrigeration. A physician's prescription may not be required. Thus, the market is expected to be unusually wide. The probiotic is expected to provide sophisticated control at a very low price.

The probiotic compositions described herein can be used to prevent or treat H. pylori infections, or diseases or disorders caused by H. pylori, in humans and animals. The probiotic compositions may be administered as a prophylactic, prior to an exposure or challenge with H. pylori. The probiotic compositions may be administered therapeutically, after an infection with H. pylori has occurred. The probiotic compositions may be incorporated into animal feed or animal drinking water.

### Example 1

Engineered proteins that specifically kill certain pathogenic bacteria without harming unrelated commensal bacteria have been developed. The specificity of killing is due to a targeting (guide) peptide attached to an antimicrobial peptide as expressed from a hybrid gene. In the present example the skin pathogen, Staphylococcus aureus, was targeted using purified guided antimicrobial protein produced from an E. coli expression system. However, the targeting system can be modified to specifically kill any bacterium.

In this example, two commonly used antimicrobial peptides (AMPs), plectasin and eurocin, were genetically fused to the targeting peptide A12C, which selectively binds to Staphylococcus species. It should be noted that A12C peptide was developed using a generic biopanning technique; in theory, any bacterium can be targeted using this method for producing guide proteins. A12C was developed by another laboratory to serve as a guide protein for vesicles, which also illustrates that peptides developed for other purposes can be repurposed to serve as guide proteins for antimicrobial peptides. The targeting peptide did not decrease activity against the targeted Staphylococcus aureus and Staphylococcus epidermidis, but drastically decreased activity against the non-targeted species, Enterococcus faecalis and Bacillus subtilis. This effect was equally evident across two different AMPs, two different species of Staphylococcus, two different negative control bacteria, and against biofilm and planktonic forms of the bacteria.

Methods:

Reagents.

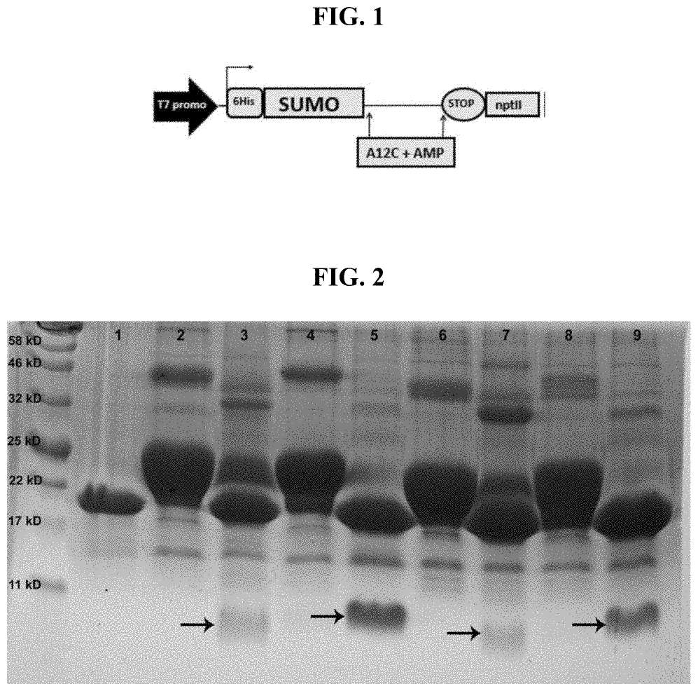

The pE-SUMOstar vector (LifeSensors) was grown in 10- and BL21 E. coli (New England Biolabs) and AMP was released from expressed fusion/AMP using Ulp1 protease produced in house. The AMPs plectasin (GFGCNGPWDEDDMQCHNHCKSIKGYKGGYCAKGGFVCKCY (SEQ ID NO:1); MW 4408) and eurocin (GFGCPGDAYQCSEHCRALGGGRTGGYCAGPWYLGHPTCTCSF (SEQ ID NO:2); MW 4345) were expressed from pE58 SUMOstar as were A12C-plectasin (MW 6137) and A12C-eurocin (MW 6074), both of which had the A12C targeting peptide (underlined) plus a short linker (GVHMVAGPGREPTGGGHM) (SEQ ID NO:3) genetically fused to the N-terminus of the respective AMP sequences. As a control, plectasin and eurocin were also conjugated with the AgrDl bacterial pheromone sequence (YSTCYFIM)(SEQ ID NO:4) (Mao et al. 2013) at the N-terminus. Synthetic A12C peptide (Biosynthesis) was used as a “target peptide only” control. FIG. 1 shows the pE-SUMOstar vector carrying AMP for expression in E. coli BL21 cells. SUMO protease site is between SUMO and A12C-AMP.

Expression, Purification and Analysis of Fusion Proteins.

The DNA sequences for the AMPs were synthesized (Integrated DNA Technologies) and ligated into the pE66 SUMOstar vector and cloned into E. coli 10-beta cells. Plasmid from these were used to transform E. coli BL21 cells for protein expression. Transformed cultures were grown out and induced with IPTG according to standard procedures. The resulting bacterial pellets were resuspended in PBS/25 mM imidazole/0.1 mg/ml lysozyme and frozen overnight. The cells were then thawed, sonicated, and ultracentrifuged at 80,000×g for 1 h at 4° C. and the 6his/SUMO/AMP fusion protein in the supernatant was purified by nickel column chromatography. The AMP was separated from SUMO by proteolysis using Ulp1 (1 U per 100 g fusion protein) at 4° C. overnight and the cleavage was evaluated by SDS-PAGE. Yields were calculated from the SDS-PAGE data, using NIH ImageJ to measure band density and the marker lane bands for mass reference. Mass spectrometry was used to ensure the proper cleavage of the AMP from the SUMO carrier protein. In-gel tryptic digest (Thermo Fisher) was performed on the AMP excised from the SDS-PAGE gel. The digest was examined by LC-ESI-MS (Synapt G2-S, Waters) at the Baylor University Mass Spectrometry Center. The analysis of the MS data was done by MassLynx (v4.1) The spectra of each protein, both non-targeted and targeted, were peak centered and MaxEnt3 processed and then matched against hypothetical peaks from peptides generated by simulated trypsin digestion of the respective proteins.

Hemolytic Activity Assay.

Guided AMPs, non-guided AMPs and synthetic A12C peptide were assessed for human hemolytic activity via exposure to washed human erythrocytes. Whole blood cells were collected a healthy volunteer using standard procedures (Evans et al. 2013) and cells were diluted in phosphate buffered saline to 5×108 cells/ml. To initiate hemolysis, 190 μl of the cells was added to 20 μl of a 2-fold serially diluted peptide/test reagent in phosphate buffered saline. Wells without peptide were used as negative controls, while wells containing 1% 85 Triton X-100 were used as positive controls.

In Vitro Bactericidal Activity Assay.

The Ulp-1 protease-cleaved proteins were tested for antimicrobial assays against four strains of bacteria: Staphylococcus aureus, Staphylococcus epidermidis, Enterococcus faecalis and Bacillus subtilis. These four species were selected because they are gram positive and the AMPs plectasin and eurocin are specifically active against gram positive bacteria (Mygind et al. 2005, Oeemig et al. 2012). The component controls were free SUMO protein and synthetically produced A12C peptide. Vancomycin was used as the positive control. The standard protocol for a microtiter plate assay with serial dilution was used in which serial 2-fold dilutions of test peptide were made across a 96-well plate containing uniform bacterial inoculum across the peptide dilutions. After bacterial growth in the presence of peptide, cell viability was assayed with resazurin. Experiments with all peptides against all bacterial species were performed with >5 replicates each.

In Vitro Cell Kinetics Study.

Ulp-1 protease-cleaved peptides were assayed to determine their dynamic action against the bacteria in a growing culture. The bacteria were grown at 37° C. with shaking and diluted to ˜1×108 CFU/ml. To these cultures were added plectasin or eurocin, at 3× the respective minimum inhibitory concentrations, or the A12C-targeted versions at these same respective concentrations. The vancomycin control concentration was the mean of the molar concentration of plectasin and eurocin used. Growth was then monitored from 2-10 h after addition of the peptides, diluting 10 μl of culture in medium and plating onto Mueller-Hinton agar plates. The number of colonies was recorded the next day.

In Vitro Biofilm Inhibition Assay.

In addition to planktonic cultures, biofilm cultures were used to assay inhibition by the peptides, using standard procedures (O'Toole 2011). Briefly, overnight cultures were diluted 1:100 and added to serially diluted peptides. Biofilms were allowed to grow for 24-36 h of unshaken culture. The liquid was removed and the biofilms were washed, dried and fixed with methanol and then stained with Crystal Violet, which was later dissolved with 30% acetic acid and the resulting solution measured for absorbance at 540 nm to quantify the amount of biofilm formed. All assays were run in triplicate or greater.

Results:

Protein Expression and Purification.

AMP/SUMO fusion proteins, with or without the A12C targeting domain, were highly expressed in E. coli BL21 cells. These were successfully cleaved with SUMO protease (Ulp-1) into their component AMP and SUMO carrier protein and were clearly visualized with SDS-PAGE as 4-6 kDa free AMP and ˜17 kDa SUMO/AMP fusion proteins. FIG. 2 shows expression of SUMO/AMP in E. coli and cleavage of AMP free of SUMO fusion partner, where Lane 1: free SUMO control and Lanes 2-9: Intact fusion proteins (even lanes) and cleaved products (odd lanes) in the following order: SUMO/plectasin, SUMO/A12C-plectasin, SUMO/eurocin, SUMO/A12C-eurocin. Arrows: free AMP The average yields (n>=3) of the proteins plectasin, A12C-plectasin, eurocin and A12C-eurocin were 15-26 mg (3-4 moles) per L of culture. For peptide confirmation, peptides were extracted from the SDS-PAGE gel bands, digested by trypsin and analyzed by mass spectrometry. Peptide identities were confirmed using the MassLynx (v4.1) application (Waters).

Hemolytic Activity Assay.

In concordance with previously published individual studies on plectasin and eurocin (Mygind et al. 2005, Oeemig et al. 2012, Yacoby et al. 2006), both guided and un-guided fusion peptides, along with the free A12C peptide control, displayed no hemolytic effect on human erythrocytes in comparison to a 20% Triton-X positive control (data not shown).

In Vitro Bactericidal Activity Assay.

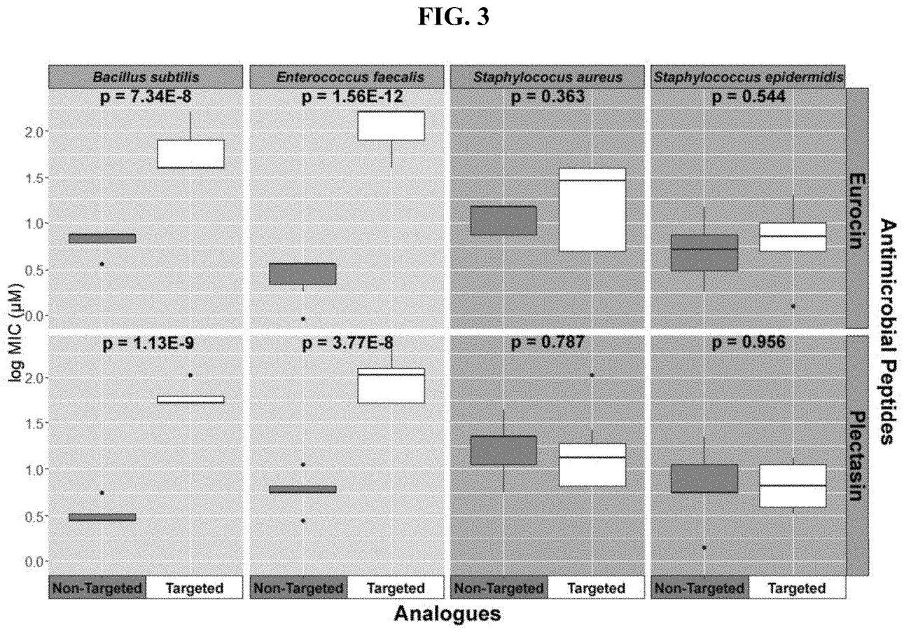

Differential toxicity against off target bacteria was observed with the A12C targeting peptide added to the AMPs. A12C-AMPs retained their toxicity against both of the targeted staphylococci bacterial species but showed a dramatic decrease in toxicity against the off target bacterial species relative to unmodified AMPs. FIG. 3 shows log values for minimum inhibitory concentrations (MIC) in μM for non-targeted and targeted analogues of eurocin and plectasin against Bacillus subtilis, Enterococcus faecalis, Staphylococcus aureus and Staphylococcus epidermidis. The boxed regions represent 50% of the values while the bars represent 95%. Unmodified plectasin and eurocin had the expected mean MIC values of 3-6 μM, which are typical values for AMPs with sequential tri-disulfide bonds produced in E. coli expression systems (Li et al. 2010, Parachin et al. 2012, Li et al. 2017). In contrast, the addition of the A12C guide peptide rendered these AMPs essentially noninhibitory to the off target bacteria, with MIC values >70 μM. In all cases, the MIC values for A12C/AMP versus AMP were significantly different for both of the off target bacteria, E. faecalis and B. subtilis (p<0.001; ANOVA 2-139 tailed test). Negative controls (SUMO alone and A12C alone) showed no antimicrobial activity (data not shown) and these were run for all experiments.

In Vitro Cell Kinetics Study.

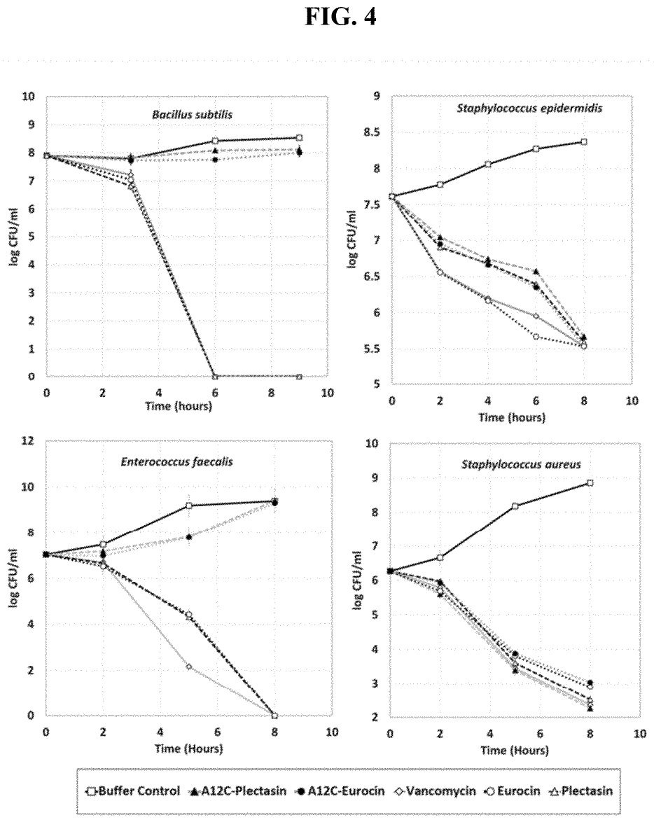

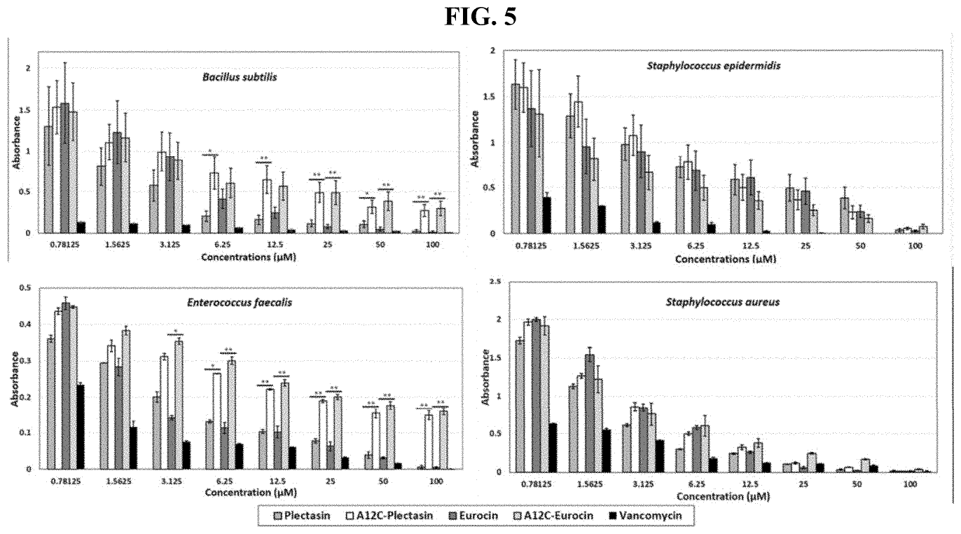

Growth kinetics over an 8 to 10 hour period more conclusively demonstrated the loss of antimicrobial activity of the A12C/AMP against the off target bacterial species. For these bacteria, A12C/AMP treatment resulted in bacterial growth that lagged only slightly behind buffer control treated cultures. FIG. 4 shows the cell-kinetic profile for B. subtilis, S. epidermidis, S. aureus and E. faecalis (clockwise), created by plotting log CFU/ml of the bacteria grown in the presence of each peptide for 8-10 hours collected in 2-3 hour intervals. Unmodified AMPs were bactericidal similar to the vancomycin control. In contrast, all peptides—both guided and unguided—demonstrated a strong bactericidal effect against the target bacteria S. epidermidis and S. aureus, similar to the vancomycin positive control. The relatively flatter growth curve for the B. subtilis control cultures reflects its growth kinetics, which is far slower than that of other bacteria.

In Vitro Biofilm Inhibition Assay.

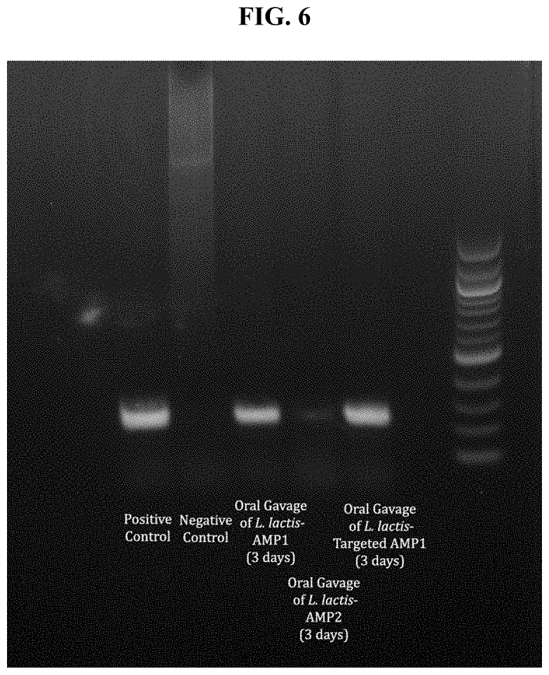

Growing bacterial cultures with the peptides demonstrated the preferential inhibition of bacterial biofilm of the Staphylococcus strains by the targeted AMPs over the non-Staphylococcus bacteria. FIG. 5 shows biofilm inhibition activity evaluated by plotting the absorbance of crystal violet (540 nm) against the concentration of 4 AMPs on the 4 bacteria—B. subtilis, S. epidermidis, S. aureus and E. faecalis (clockwise). (*=p<0.1, **=p<0.05, n>=3). The absorption reading (hence, the quantity of biofilm formed) decreased with the increase in peptide concentration for all the 4 bacteria when treated with unguided peptides but the guided peptides did not have similar effects on B. subtilis and E. faecalis with significant (p<0.10 or p<0.05) difference in the absorbance values between targeted and non-targeted AMPs at concentrations beyond 6.25 μM.

This example demonstrates successful targeting of the AMPs plectasin and eurocin against two staphylococcal bacteria. Importantly, this was achieved by essentially eliminating the activity against the two off target bacteria tested. This is the expected outcome for an antimicrobial therapy that preserves the commensal members of the microbiome while killing the pathogenic target bacteria. This is also the outcome that was achieved against S. aureus by Mao et al. (2013) with the use of a bacterial pheromone peptide for targeting of plectasin. Other than a lower MIC for the unmodified plectasin itself, the same drastic degree of reduction in the activity against the off target bacteria, E. faecalis and B. subtilis was seen, as was reported by Mao et al. (2013). Thus, it is demonstrated that a biopanning-derived ligand works as efficiently as a pheromone-derived ligand, which is the class of targeting peptide used in all targeted AMPs to date. It should be noted that the pheromone-derived ligand was more specific than A12C, with activity against S. aureus but not S. epidermis, while A12C/plectasin was highly active against both species.

Four main sources of ligands exist for use as guide peptides for AMPs. First, bacterial pheromones are species-specific peptide signals which trigger the development of competence, virulence, or other capabilities, and pheromone peptides have been determined for many pathogenic bacteria (Monnet et al. 2016). Second, biopanning is a means of screening random libraries of peptides for the ability to bind to a target sequence, such as a receptor on a bacterial cell. Usually, a bacteriophage is used to display the members of the peptide library (Wu et al. 2016). Third, bacteriophage receptor binding proteins can be used as a resource for the development of targeting peptides for AMPs. The receptor binding proteins of phages against many pathogenic bacteria have already been characterized (Dowah and Clokie 2018, Nobrega et al. 2018). In addition, screens for new phages against lesser studied bacterial pathogens can be carried out (Weber-Da̧browska et al. 2016). Fourth, virulence factors of the targeted bacterial pathogen can be targeted by using targeting (guide) peptides consisting of the sequence of the host receptor that is bound by the bacterial virulence factor. In this way, the host receptor sequence is used as a guide peptide to direct an AMP back to the bacterial pathogen. This is demonstrated in the experiments of this patent application.

### Example 2

An exemplary probiotic bacterium, Lactococcus lactis, has been shown to survive well in the stomach of mice. Mice were force fed the recombinant probiotic by oral gavage and recombinant bacterial DNA was recovered from the stomachs of the mice a full 3 days after introduction. In FIG. 6, it is seen that Lactococcus lactis harboring the pT1bin1 expression vector with the open reading frames of either the antimicrobial peptide laterosporulin (AMP1) or with the antimicrobial peptide alyssaserin (AMP2) or with laterosporulin genetically fused to the guide peptide open reading frame derived from multimerin (targeted AMP1) were all present 3 days after the introduction of these bacteria to the mice by oral gavage, as evidenced by PCR (using vector-specific primers) of the stomach reverse gavage extracts. This indicates that recombinant Lactococcus lactis was thriving in the stomachs of the mice. Force feeding (oral gavage) was used to ensure that a consistent amount of bacterium was delivered to each mouse. Reverse oral gavage was used to flush mouse stomach with buffer and collect the stomach contents for PCR analysis. In FIG. 6, the Positive Control was PCR of the pT1bin1/laterosporulin DNA and the Negative Control was PCR of no template DNA, with the same vector-specific primers used in both of these control PCRs as was used for the PCRs for the mouse extracts in the other lanes. The last lane of FIG. 6 is a marker lane with a DNA ladder. All positive bands comprised DNA of the expected size.

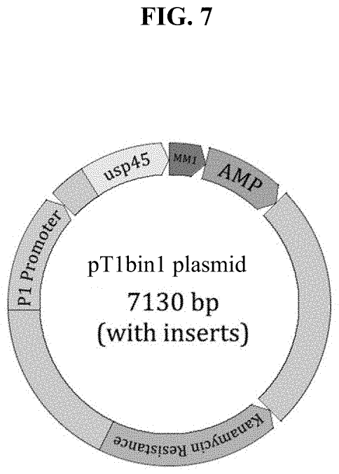

A vector has also been developed that greatly facilitates Lactococcus lactis engineering. To create this vector (shown in FIG. 7), the original Lactococcus lactis vector, pT1NX, was modified by the addition of and E. coli origin of replication and a kanamycin resistance cassette, both from the SUMO-based E. coli expression vector, pE-SUMOstar. In FIG. 7, the kanamycin resistance block represents both the kanR cassette and the E. coli origin of replication. This binary vector (pT1bin1) can be grown in E. coli to facilitate the addition of AMP or guide sequence inserts by recombinant DNA techniques. Generous quantities of plasmid can be produced via standard plasmid preparation techniques in order to ease the transformation of Lactococcus lactis. This latter transformation is difficult to achieve with ligation products, but is easier with DNA from plasmid preparations.

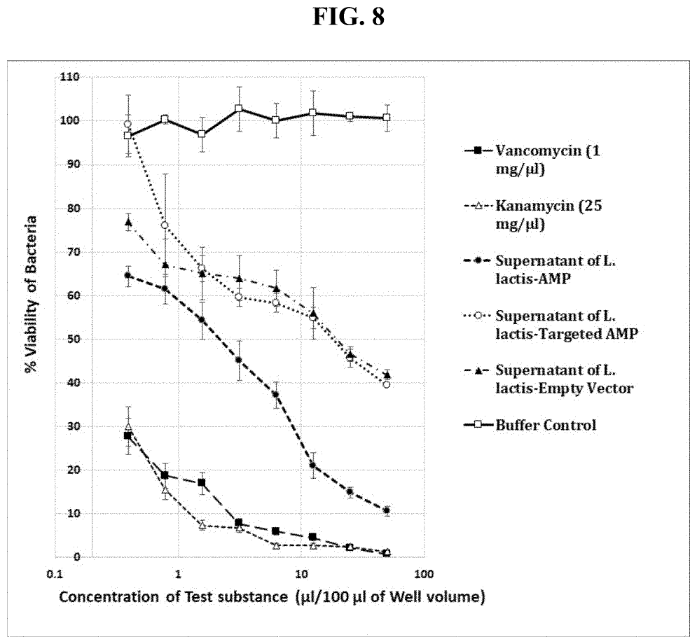

It has been demonstrated in vitro that engineered Lactococcus lactis secreting antimicrobial peptide kills other bacteria in vitro. This is reported in FIG. 8 as the survival of E. coli in the presence of broth culture of Lactococcus lactis secreting antimicrobial peptide with or without a guide peptide. FIG. 8 shows the viability of E. coli in the presence of different antibiotic dilutions and supernatants. It should be noted that the legend is in reverse order of the lines, top to bottom, with the upper line in the graph being the buffer control and the lower line being vancomycin. To obtain the results shown in FIG. 8, cultures of Lactococcus lactis containing either the empty pT1bin1 vector, pT1bin1 harboring the antimicrobial peptide laterosporulin, or pT1bin1 harboring laterosporulin genetically fused to the guide peptide from multimerin were centrifuged to remove bacterial cells and the resulting supernatants were added to separate starter cultures of E. coli to check for inhibition of E. coli growth. The starter culture used supplying all replicates consisted of 500 μl of overnight culture of E. coli diluted in 50 ml of LB broth. Three replicates of each treatment were conducted and each point in the graph represents an average with corresponding error bars. To run the treatments and replicates, a 96-well microtiter plate was used. For each well, 100 μl of diluted Lactococcus lactis supernatant was added to 100 μl of E. coli starter culture. As seen in the x-axis of FIG. 8, the dilutions used ranged from no dilution (100 μl of 100% supernatant added to the 100 μl of E. coli) down to 1/200 dilution of supernatant (100 μl of 0.5% supernatant added). Antibiotic positive controls were diluted similarly, with the starting concentrations (undiluted) stated in the legend. The y-axis of FIG. 8 represents the inhibition of E. coli viability by these supernatant and antibiotic dilutions. E. coli viability was measured by plating onto LB agar plates the cultures in each well after 4 hours of exposure to supernatant or antibiotic. The resulting colonies appearing on the plates were recorded, with the undiluted buffer control treatment being set to 100% and all other treatments being converted to a fraction of this value, as plotted on the y-axis.

Looking at FIG. 8, it can be seen that the buffer control did not inhibit E. coli. However, Lactococcus lactis broth culture (with cells removed) did inhibit E. coli even with no recombinant antimicrobial peptide present (empty vector control). This is considered the baseline for examining the effect of the secreted recombinant proteins. The expression of laterosporulin by Lactococcus lactis resulted in a significant decrease in viability of E. coli compared to this baseline. However, there was no significant difference seen between the empty vector baseline and the multimerin-guided (targeted) lactosporulin. This means that the guide peptide completely abolished antimicrobial activity of laterosporulin against the nontarget bacterium E. coli. This is in agreement with results shown in Example 1 with Staphylococcus. This data supports the ability of these extracts to kill different target bacterium, such as Helicobacter pylori.

### Example 3

In Vitro Control of Helicobacter pylori by Lactococcus lactis Expressing gAMPs.

Purpose

This example demonstrates that antimicrobial peptide (AMP) fused to the multimerin-derived guide peptide specific for Helicobacter pylori, expressed from a hybrid gene and secreted from the probiotic Lactococcus lactis, can specifically kill H. pylori when the probiotic is co-cultivated with H. pylori in vitro. This was an in vitro proof of principle before conducting the in vivo studies in mice.

Experimental Design

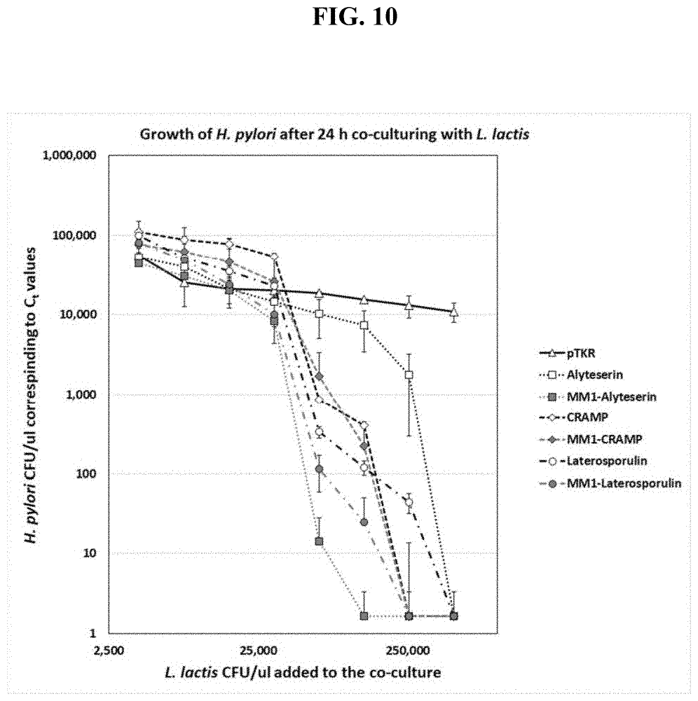

In the co-cultures, different dilutions of L. lactis were used but each well had 10 μl of H. pylori culture (˜3000 CFUs). The L. lactis secreted AMP, gAMP or contained an empty expression vector. Alyteserin and CRAMP were the AMPs tested. These were constructed either genetically fused to the multimerin-derived guide peptide (guide AMP or gAMP) or not (AMP). The amount of H. pylori present in the co-culture at any given time point was measured by qPCR, using primers specific for the VacA gene itself, which codes for the receptor protein to which the gAMP binds. The entire experiment was run in triplicate and the growth of H. pylori after 24 h in the presence of L. lactis expressing various AMPs or gAMPs is shown in FIG. 10.

Methods

Genetic Constructs

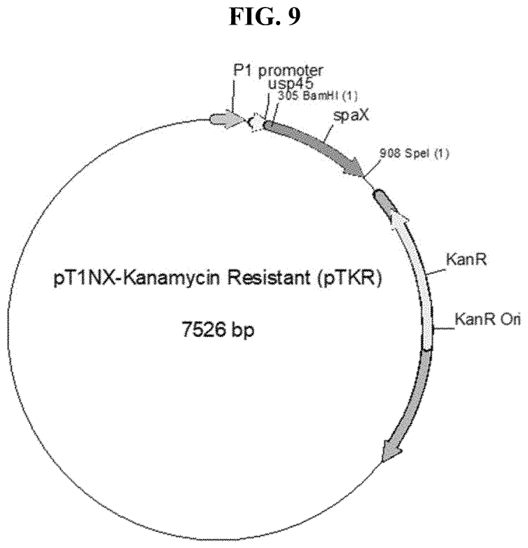

FIG. 9 shows the vector for Lactococcus lactis secretion of AMPs and gAMPs. The ORFs of the AMPs, codon-optimized for Lactococcus lactis, were cloned into the modified pT1NX-kanR (pTKR) vector for L. lactis expression/secretion in between the restriction enzyme sites BamHI and SpeI by replacing the spaX protein of the original plasmid. The P1 promoter upstream of the BamHI cut-site controls the downstream expression as a constitutive promoter which is upregulated by low pH. The usp45 gene immediately upstream of BamHI site codes for an endogenous signal peptide of L. lactis that allows secretion of the resulting fusion peptide. After ligation of the AMP/tAMP into pTKR vector, it was transformed into E. coli (10β, NEB) and plated onto kanamycin selective plate. The pT1NX plasmid (LMBP 3498) has erythromycin resistance but was modified to create pTKR as shown in FIG. 9, which also has kanamycin resistance for cloning into electrocompetent E. coli (10β, NEB) for plasmid propagation. Extracted plasmid from the E. coli was then electroporated into electrocompetent L. lactis MG1363 (LMBP 3019) and plated on erythromycin selective GM17 plates (30° C., microaerobic, overnight). After screening for the presence of the AMP/gAMP ORFs with PCR, selected colonies were propagated in liquid cultures of M17 broth with glucose (0.5% w/v) in the presence of erythromycin (5 μg/ml).

AMP/gAMPs Used in this Experiment

The following AMPs and guided AMPs (gAMPs) were cloned into the secretion vector pTKR. The multimerin1 (MM1) guide peptide sequence MQKMTDQVNYQAMKLTLLQK (SEQ ID NO:5) is underlined and the serine/glycine linker sequence is in bold.

| AMP/gAMP | Peptide Sequence |
|---|---|
| Laterosporulin | ACQCPDAISGWTHTDYQCHGLENKMYRHVYAICMNGTQVYCRTEWGSSC |
| | (SEQ ID NO: 6) |
| MM1- | MQKMTDQVNYQAMKLTLLQKSGGGSACQCPDAISGWTHTDYQCHGLENK |
| Laterosporulin | MYRHVYAICMNGTQVYCRTEWGSSC (SEQ ID NO: 7) |
| Alyteserin | GLKDIFKAGLGSLVKGIAAHVAN (SEQ ID NO: 8) |
| MM1- | MQKMTDQVNYQAMKLTLLQKSGGGSGLKDIFKAGLGSLVKGIAAHVAN |
| Alyteserin | (SEQ ID NO: 9) |
| CRAMP | ISRLAGLLRKGGEKIGEKLKKIGQKIKNFFQKLVPQPE (SEQ ID NO: 10) |
| MM1-CRAMP | MQKMTDQVNYQAMKLTLLQKSGGGSISRLAGLLRKGGEKIGEKLKKIGQKIK |
| | NFFQKLVPQPE (SEQ ID NO: 11) |

Underline = Multimerin1,
Bold = linker

S L. lactis/H. Pylori Co-Culture and qPCR Analysis

L. lactis AMP/gAMP clones were propagated from glycerol stocks and grown in GM17 broth overnight with erythromycin (5 μg/ml) with no shaking. H. pylori stocks were first propagated on Blood-TS agar overnight with microaerobic condition and >5% CO₂ environment. Then colonies from the plate were transferred to a TS broth with newborn calf serum (5%) and grown overnight under microaerobic condition and >5% CO₂ environment. The L. lactis cultures were serially diluted in a 96-well culture plate with TSB broth to make up a volume of 100 μL. To each well, 10 μL of the overnight H. pylori culture was added and each well volume was brought up to 200 μL with more TS broth. The plate was left to grow overnight in a microaerobic environment with >5% CO₂. After 24 h, well contents from the culture plate were transferred to a 96-well PCR plate. That PCR plate was sealed and heated for 15 min at 100° C. and chilled at 4° C. for 5 min. Then the plate was centrifuged at 2000 g for 2 min and the supernatant was used as the template for qPCR. The qPCR was done using primers for VacA gene to quantify H. pylori (forward: 5′-ATGGAAATACAACAAACACAC-3′ (SEQ ID NO:12), reverse: 5′-CTGCTTGAATGCGCCAAAC-3′ (SEQ ID NO:13) and primers for acma gene for quantifying L. lactis. Standard curves for H. pylori and L. lactis were constructed by determining C_{T} values for different dilutions of the overnight cultures of the respective bacteria (1/10, 1/100, 1/1000, 1/10000) in the qPCR plates, the CFUs for the dilutions were determined by plating on their respective agar plates.

Results

FIG. 10 shows the results of qPCR on VacA gene of H. pylori co-cultured with L. lactis expressing gAMPs or AMPs. L. lactis expressing AMPs with or without guide peptides knocked down the H. pylori culture to below the baseline of detection for this experiment (C_{T} value of 40). Plain AMPs are represented with open symbols while gAMPs are represented with solid gray symbols. Alyteserin was not very effective unless fused to the guide peptide. The control experiment (solid line), with L. lactis carrying the empty vector, showed that the L. lactis probiotic, by itself, had little to no influence on the growth of H. pylori over 24 hours. Error bars represent 95% confidence limits.

Conclusions

L. lactis expressing two different AMPs was able to knock down, to baseline levels, a vigorous H. pylori culture in vitro. The multimerin guide peptide sequence was shown to not interfere with AMP toxicity in CRAMP, with targeted and untargeted CRAMP equally toxic to H. pylori. In all cases, the gAMP (“MM1” prefix) was more toxic (lower on y-axis) than the corresponding AMP. In the case of the alyteserin AMP/gAMP pair, the guide peptide appeared to be a requirement for high toxicity to H. pylori.

### Example 4

Effect on Off-Target Bacteria of Probiotic gAMPs.

Purpose

To determine the effect of L. lactis probiotic expressing AMP or gAMP on off-target bacteria in vitro.

Experimental Design.

The experimental design was identical to that described above in Example 3, with the exception of the off-target bacterium replacing the targeted H. pylori. The off-target bacteria used were Lactobacillus plantarum (gram positive) and Escherichia coli (gram negative).

Methods

All methods are described above in Example 3.

Results

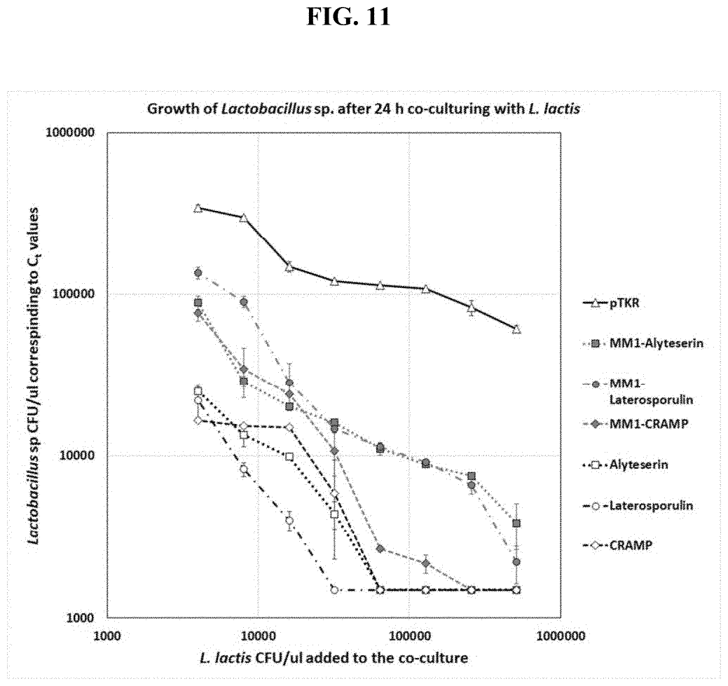

FIG. 11 shows growth of Lactobacillus plantarum after 24 hours co-culturing with L. lactis expressing empty vector (pTKR), AMPs (alyteserin, laterosporulin, or CRAMP), or gAMPs (MM1-alyteserin, MM1-laterosporulin, or MM1-CRAMP). Compared to the probiotic only control (Lactococcus lactis with empty vector pTKR), cocultivation of Lactobacillus plantarum with probiotic expressing either AMP or gAMP led to a reduction in off-target titer with increasing amounts of probiotic deployed. However, there was significantly more negative effect on off-target growth by probiotic/AMP treatment than with probiotic/gAMP treatment for all three AMPs tested. Specifically, at the 100,000/μl CFU level which was was maximally efficacious for H. pylori kill in Example 3, all probiotic/AMP treatments led to Lactobacillus levels undetectably low by qPCR. In contrast, probiotic/gAMP levels were at 10,000 CFU/μl for alyteserin and laterosporulin gAMPs and 2500 for CRAMP gAMP. At lower probiotic levels, a 5 to 7-fold differential occurred between gAMP and AMP probiotic treatment, with probiotic/gAMPs significantly less deleterious to off-target Lactobacillus than probiotic/AMPs. Error bars represent 95% confidence limits.

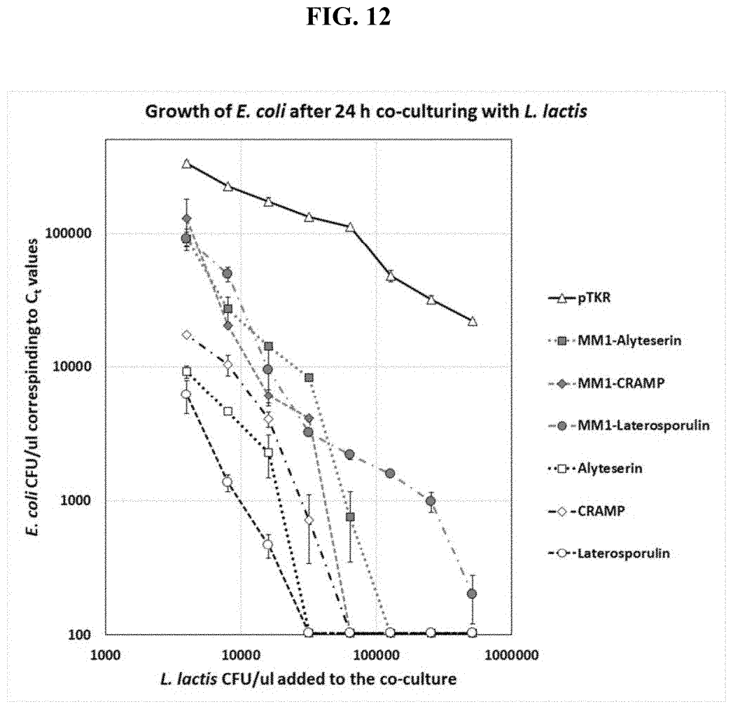

FIG. 12 shows growth of Escherichia coli after 24 hours co-culturing with L. lactis expressing empty vector (pTKR), AMPs (alyteserin, laterosporulin, or CRAMP), or gAMPs (MM1-alyteserin, MM1-laterosporulin, or MM1-CRAMP). As seen in FIG. 12, results were similar for off-target effects against E. coli as they were for L. plantarum described above.

Conclusions

It can be concluded from these in vitro off-target results that gAMPs are significantly less deleterious to these two off-target bacterial examples than AMPs in aprobiotic delivery system. These in vitro results show at least a portion of the picture of off-target effects of probiotic AMPs and gAMPs. As discussed more below, averaged across the entire mouse stomach microbiota, the probiotic gAMPs have no more disruptive effect than unengineered Lactococcus lactis probiotic.

### Example 5

Therapeutic Control of H. pylori in Mice.

Purpose

The control of H. pylori by Lactococcus lactis expressing gAMPs was tested in vivo in mouse. A therapeutic test is more stringent than a prophylactic test since the pathogen is given time to establish and replicate in the mouse before the probiotic is introduced. This most stringent test was chosen to evaluate the effect of different AMPs, testing three different AMPs, in guided and unmodified forms.

Experimental Design

Probiotic Control Mice.

These mice received only the probiotic, prepared as described in the previous example. Stomach samples were collected on Day 0 before inoculation with reverse-oral gavage; resuspended L. lactis were fed to the mice by oral gavage; stomach samples were taken on Days 3, 5 and 7.

Therapy Treatment of H. pylori-Infected Mice.

These mice were inoculated with H. pylori and the H. pylori was allowed to establish itself in the mouse stomach for 3 days, with daily inoculations to ensure establishment. The mice were then given L. lactis secreting AMP or gAMP to therapeutically treat the H. pylori infection. Stomach samples were collected on Day 0 before H. pylori inoculation; resuspended H. pylori were fed by oral gavage once daily for 3 consecutive days; stomach samples were then collected on Day 5 to test for H. pylori presence and on Day 5 resuspended L. lactis were fed to the mice; subsequent stomach samples were collected on Day 8 and 10.

Untreated H. pylori Infection Control Mice.

These mice were infected with H. pylori and no prophylatic or therapy was provided. Stomach samples were collected on Day 0 before H. pylori inoculation; resuspended H. pylori were fed by oral gavage once daily for 3 consecutive days; stomach samples were then collected on Day 5, 8 and 10 to test for H. pylori presence.

Methods

Administering L. lactis and H. pylori in Mice by Oral Gavage.

The L. lactis cultures were propagated overnight as described above. The overnight cultures were spun down at 4000 g for 15 min at 4° C. The pellets were resuspended in sterile PBS. H. pylori stocks were grown overnight on Blood-TS agar as described above and then scraped by a sterile loop and resuspended in sterile PBS. The either bacterial suspension were fed to the mice using 1.5 ga oral gavage needle not exceeding half their stomach volume (˜250 μL). The colony forming units (CFUs) of the resuspension being fed were determined by diluting the resuspension 1/1000 and 1/10000 times and plating on appropriate plates. Pre- and post-inoculation samples from the mouse stomach were collected by flushing their stomach with excess PBS (˜300 μL) and the stomach fluid was collected by reversing the oral gavage injection until the vacuum was maintained.

Assay for H. pylori and L. lactis Presence by qPCR.

The stomach samples collected were heated at 100° C. for 15 min and chilled at 4° C. for 5 min. The supernatants were collected and plated in a 96-well plate and qPCR was performed with primers for the VacA gene to quantify for H. pylori and primers for the acma gene to quantify for L. lactis. Standard curves for each bacterium against their CT values were constructed by including different dilutions of the overnight cultures of the respective bacteria (1/10, 1/100, 1/1000, 1/10000) in the qPCR and plating those dilutions on respective plates to determine the corresponding CFU values. Each data point represents at least 3 replicate mice.

qPCR Value Standardization.

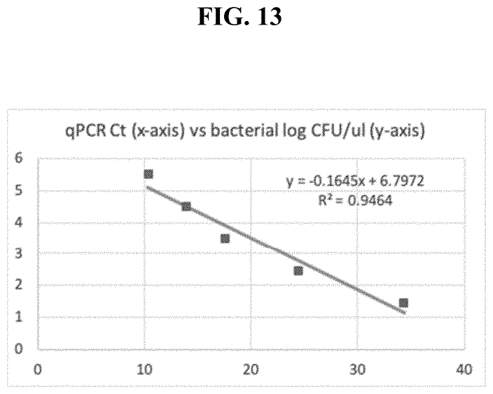

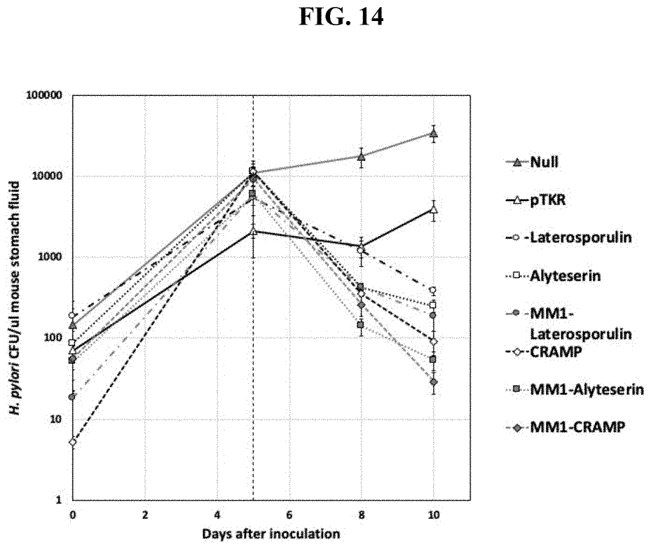

The standard curve for CFU/μl of H. pylori culture with CT values is shown in FIG. 13. This was used to generate CFU/μl data from qPCR CT values in FIG. 14.

Results

Before inoculation with H. pylori, at Day 0, mice had very low levels of native H. pylori with the VacA gene (8-200 CFU/μl) (FIG. 14). At 5 days after inoculation with H. pylori, 2,000-12,000 CFU/ul of H. pylori was recorded, indicating strong replication in the mouse stomach. At Day 5, mice were inoculated with probiotics, except for the Null control. H. pylori continued replicating well in the Null control mice, increasing 3-fold after Day 5 and reaching 40,000 CFU/μl at Day 10. After probiotic therapy treatment at Day 5, the pTKR (empty vector) probiotic control increased 2-fold to Day 10. In the mice used for pTKR treatment, the H. pylori inoculation was not as effective and thus the H. pylori titer was lower at Day 5 than the other mouse groups, even before probiotic treatment.

In contrast, all mice given probiotics expressing AMP or gAMP experienced a strong decline in stomach H. pylori after probiotic therapy delivered at Day 5 (FIG. 14). This decline was between 15-fold and 320-fold depending on the AMP or gAMP treatment, which led to final H. pylori levels 100 to 1000-fold less than the Null control, which received no probiotic therapy and had continued H. pylori growth after Day 5. Furthermore, within each of the three AMP/gAMP pairs, the AMP treatment was significantly less effective at controlling H. pylori than the gAMP treatment. Specifically, at Day 10, for alyteserin and CRAMP, there was 15-fold more H. pylori with the AMP versus the gAMP, while for laterosporulin, there was 2.5-fold more H. pylori for the AMP versus the gAMP. Error bars represent 95% confidence limits.

FIG. 14 shows the CFU/μl of H. pylori in mouse stomach fluid for control mice (Null) and mice inoculated with empty vector (pTKR) or Lactococcus lactis probiotic secreting AMPs or gAMPs (where MM1=Multimerin1 guide peptide).

Conclusions

The expression of AMP or gAMP in the probiotic L. lactis led to significant reduction (15 to 320-fold) in H. pylori titers in mouse stomach previously inoculated with H. pylori. Thus, probiotic L. lactis engineered to express AMP or gAMP can be expected to serve as a strong therapeutic treatment for H. pylori. Furthermore, a significant distinction can be drawn between AMP and gAMP effector proteins, and this differential holds up for all three AMPs tested. gAMPs were 2.5, 15, and 15-fold more effective at eliminating H. pylori than AMPs for laterosporulin, alyteserin, and CRAMP, respectively. Thus, gAMP technology is functionally superior in killing efficacy to AMP technology when delivered via probiotics for application against H. pylori.

### Example 6

Prophylactic Control of H. pylori in Mice.

Purpose

Mice were inoculated with the probiotic, L. lactis, secreting gAMP or AMP as a prophylactic treatment in order to prevent the establishment of H. pylori after challenge inoculation 3 days later. Though any medical application of probiotic gAMP technology would be expected to be therapeutic rather than prophylactic, and though this is a less stringent test of effectiveness than the therapeutic test, this experiment was run for completeness.

Experimental Design

Probiotic Control Mice.

These mice received only the probiotic, prepared as in the examples above. Stomach samples were collected on Day 0 before inoculation with reverse-oral gavage; resuspended L. lactis were fed to the mice by oral gavage; stomach samples were taken on Days 3, 5 and 7.

H. pylori Challenge to Probiotic Prophylactic Treatment of Mice.

These mice received a probiotic expressing AMP or gAMP and then were challenged 3 days later with H. pylori. Stomach samples were collected on Day 0 before L. lactis inoculation; resuspended L. lactis were fed by oral gavage on the same day; stomach samples were then collected on Day 3 to test for L. lactis presence and on Day 3 resuspended H. pylori were fed to the mice once daily for 3 consecutive days; subsequent stomach samples were collected on Day 8 and 10.

Untreated H. pylori Infection Control Mice.

These mice were infected with H. pylori and no prophylatic or therapy was provided. Stomach samples were collected on Day 0 before H. pylori inoculation; resuspended H. pylori were fed by oral gavage once daily for 3 consecutive days; stomach samples were then collected on Day 5, 8 and 10 to test for H. pylori presence.

Results

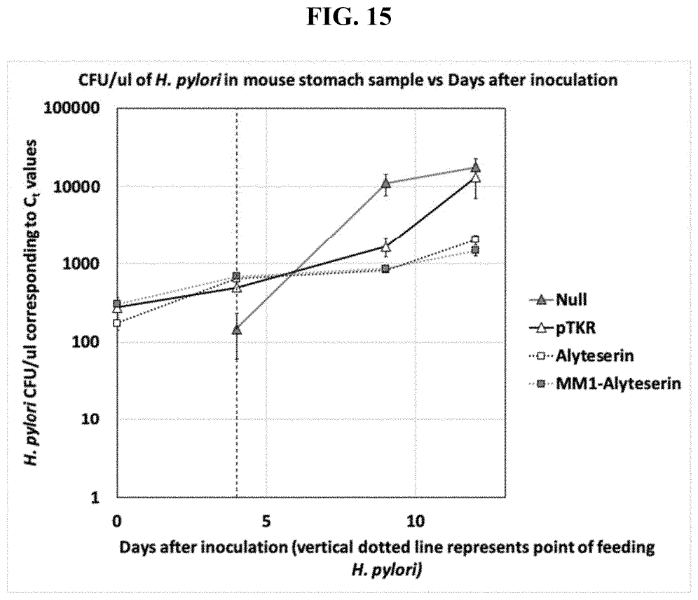

FIG. 15 shows the CFU/μl of H. pylori in mouse stomach fluid for control mice (Null) and mice inoculated with empty vector (pTKR) or Lactococcus lactis probiotic secreting AMPs or gAMPs (where MM1=Multimerin1 guide peptide), followed by additional feeding of H. pylori. All mice started with 170-300 CFU/μl of native H. pylori at Day 0, before L. lactis inoculation. By Day 4, native H. pylori had increased to 500-700 CFU/μl just before exogenous H. pylori challenge. By Day 12, H. pylori had increased only to 1500 (gAMP) and 2000 (AMP) CFU/μl in the probiotic prophylactic mice treated with MM1-Alyteserin (gAMP) or Alyteserin (AMP). In contrast, H. pylori increased to 13,000 and 18,000 CFU/μl in mice given a prophylactic pre-treatment with empty vector (pTKR) or no prophylactic probiotic, respectively. Error bars represent 95% confidence limits.

Conclusions

Probiotics engineered to deliver AMP or gAMP both provided strong prophylactic protection against H. pylori challenge. H. pylori increased only 2-fold in 7 days after H. pylori challenge with probiotic/AMP or probiotic/gAMP pre-treatment. In contrast, with empty vector probiotic, H. pylori increased 26-fold in 7 days. This demonstrates that prophylactic treatment is very effective against H. pylori infection.

### Example 7

Microbiome Sequence Analysis Demonstrates Only Slight Disruption to the Mouse Stomach Microbiota

Purpose

The stomach microbial communities of mice from the prophylactic and therapeutic experiments were examined by next generation sequencing. The effect of these treatments on the microbial diversity in the stomach will be determined. It was expected that, due to the selective toxicity of gAMPs, the microbiota of the probiotic/gAMP-treated mice will be more diverse than that of the probiotic/AMP-treated mice.

### BACKGROUND

H. pylori has been found to cause dysbiosis of the gut microbiota in humans (Liou et al., 2019). In humans, it has been found that gut microbial diversity decreases with increasing H. pylori infection while the eradication of H. pylori is often associated with an increase in microbial diversity (Li et al., 2017). However, antibiotic treatment, in general, is associated with a decrease both taxonomically and in terms of bacterial abundance in the gut (Lange et al., 2016). In this study, mice treated with H. pylori were given a variety of therapeutic treatments at Day 5 and then compared. In this way, a comparison of the effect of H. pylori infection on taxonomy versus infection treated with probiotic alone, probiotic/AMP, probiotic/gAMP, or antibiotics was possible.

Experimental Design

The experiments for the therapeutic and prophylactic studies generated mouse reverse-oral gavage samples that were used for qPCR in Examples 5 (therapy) and 6 (prophylactic) above. These same samples were analyzed for population shifts in the stomach microbiota using next generation sequencing. Hence, the experimental design is identical to Examples 5 and 6.

Methods

As described for Examples 5 and 6, the mouse stomach samples collected by reverse oral gavage were heated at 100° C. for 15 min and chilled at 4° C. for 5 min. The supernatants were collected and plated in 96 well plate for upstream processing for Next Gen sequencing. The samples were amplified with 16s primers and then with Illumina index primers with subsequent clean-up and purification. The samples were pooled into a library and sequenced using Illumina MiSeq v3 kit. The data was demultiplexed, denoised and analyzed using QIIME2.

Results

Effects of Therapeutics on Stomach Total Bacterial Diversity: Rarefaction Estimates.

Rarefaction curves derived from Illumina MiSeq next generation sequencing were used to estimate total bacterial abundance. These represent the number of species (operational taxonomic units, OTUs) that were detected within different portions of the data set. The different portions of the data set are randomly chosen subsamples. Rarefaction curves were used to determine the minimum number of samples that can be used while still representing the entire range of OTUs in order to reduce computer load in calculations. For our purposes, this standard graphic reveals the species diversity from each treatment.

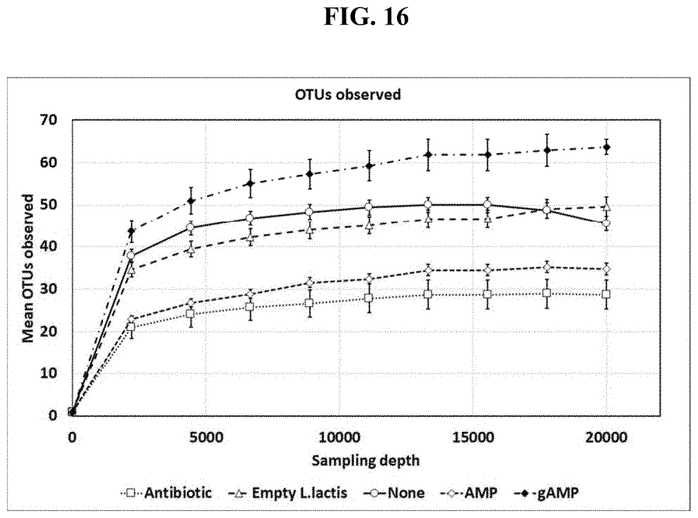

Striking differences in bacterial diversity were observed in data from Day 8 and 10 of the therapeutic study detailed in Example 5 (FIG. 16). In this study, H. pylori infection had developed for 5 days by Day 5. On Day 5, the therapy was administered. By Days 8 or 10, the therapy had 3 or 5 days to affect the stomach microbiota, respectively. As shown in FIG. 16, the use of the combination antibiotic tetracycline/amoxicilin resulted in the lowest species diversity. Importantly, the use of AMPs delivered by the probiotic (data from all three AMPs represented here) led to less diversity compared to the use of probiotic with the empty vector or no therapy, but more diversity compared to the use of antibiotics. The maximal diversity resulted from treatment with the probiotic expressing gAMP (data from all three AMPs represented in FIG. 16).

The differential seen in the in vitro experiments of Example 2 in terms of off-target effects was likely seen at a broad scale in this in vivo data. With reduced off-target effects, the expression of gAMP by probiotics led to a broader range of bacterial species surviving compared to AMPs. It is likely that lower diversity seen with probiotic/empty vector or no therapy was due to their ineffectiveness in killing H. pylori, which has been shown to reduce bacterial diversity in previous studies (Lange et al., 2016). Even though AMPs and antibiotics are able to kill H. pylori, their own broad scale toxicity was seen here to decrease bacterial diversity.

Effects of Therapeutics on Indicator Species

There are only a few publications identifying mouse stomach bacteria as beneficial to the gut microbiota. Muribacter muris (syn. Actinobacter muris) is a common mouse commensal bacterium and has been used as a niche replacement for the successful elimination of the pathogen Haemophilus influenzae in mice resulting in lowered inflammation (Granland et al., 2020). Lactobacillus murinus, a predominant mouse gut commensal bacterium, has been shown to reduce gut inflammation (Pan et al., 2018). Lactobacillus reuteri has been shown to stop autoimmunity in mouse gut (He et al., 2017) and has been used to protect mice against enterotoxigenic E. coli infection (Wang et al., 2018) and also has been shown to have anti-inflammatory effects in humans in many studies (Mu et al., 2018). Since all of these species were found to predominate in our next generation sequencing results we used them as indicator species for a healthy gut microbiota.

In order to pick microbiota dysbiosis indicators, the two bacterial genera among the top 10 most abundant bacteria that spiked during H. pylori infection were chosen, namely, Staphylococcus and Acinetobacter.

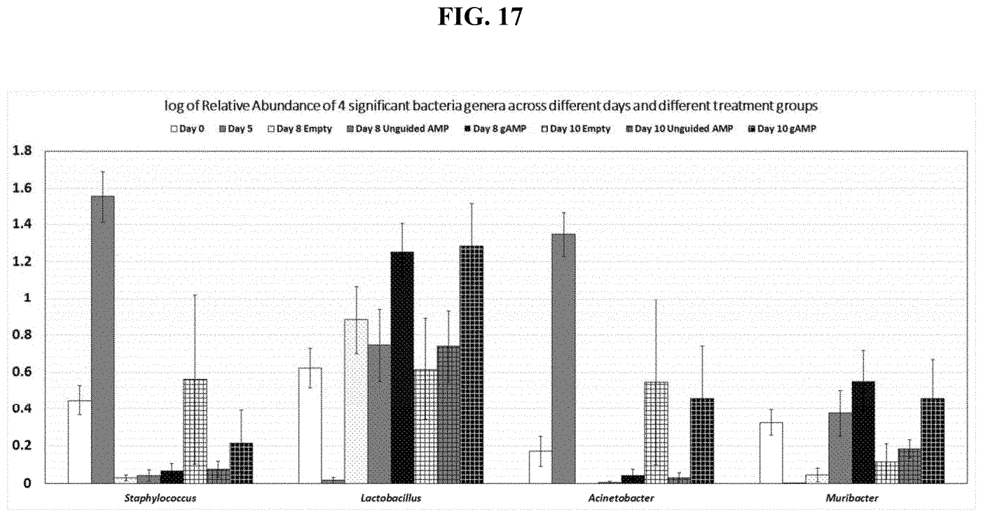

The abundances of these bacteria, as determined from next generation sequencing data, are shown in FIG. 17 for various time points and treatments. It can be seen that the beneficial indicators, Lactobacillus and Muribacter, both decreased in response to H. pylori infection, but rebounded to levels greater than pre-infection levels after treatment with probiotic/gAMP. This rebound effect was greater than seen with probiotic/AMP. For the dysbiosis indicators, both Staphylococcus and Acinetobacter increased greatly in abundance in response to H. pylori infection, but were greatly reduced in response to probiotic expressing either gAMP or AMP.

It is difficult to analyze the thousands of bacteria detected by next generation sequencing in the mouse stomach in these therapeutic experiments. Furthermore, it is difficult to examine such single-species data given the paucity of published information concerning the benefit or detriment of single bacterial taxa on mouse stomach microbiota populational health. However, these four bacteria do have significance to mouse stomach microbiota health and the effect of probiotics expressing gAMPs on these four indicator species supports our general hypothesis of the beneficial effects of probiotic/gAMP treatment. Probiotic/gAMP treatment increased the abundance of the known beneficial indicator species and decreased the most abundant dysbiosis indicator species following therapy of H. pylori infection.

Effects of Treatments on Noninfected Mice Over Time

The effects of various therapeutic treatments over time on noninfected mice is an important question to ask. Any therapy or prophylactic treatment should have as minimal negative impact on the native gut flora as possible. As a standard treatment baseline, it has been shown that antibiotics have a devastating effect on gut microbial diversity (Lange et al., 2016). Both the infection by H. pylori and the therapeutic elimination of H. pylori are expected to be negative and positive confounding factors, respectively, in terms of diversity evaluation (Liou et al., 2019; Li et al., 2017). Thus, the proper experimental design would not include H. pylori. For this reason, the effects of the various the therapeutic treatments on uninfected mice were compared.

The mouse stomach microbiota consists of thousands of species of bacteria. In order to depict changes in number in each of these species that occur before and after treatment, it is necessary to use certain statistical indices. The following indices indicate that gAMP treatment causes far less change to the stomach microbiota than AMP treatment.

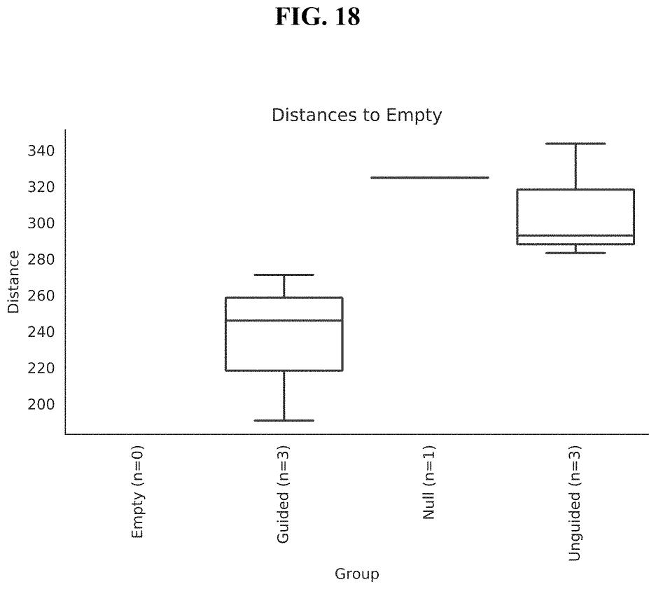

In FIG. 18, all of the bacterial species from mouse stomach are compared between four treatment groups: Empty (probiotic carrying only an empty vector), Null (mock inoculation with buffer), Guided (probiotic expressing gAMP), and Unguided (probiotic expressing AMP). In this figure, the latter three treatments are compared to the Empty treatment. Generally speaking, the y-axis represents the taxonomic distance of the collection of bacterial species in each treatment compared to the collection of bacterial species in the Empty treatment. Specifically, the index used (y-axis) is a plugin from QIIME called the Nonparametric Microbial Interdependence Test (NMIT) (Zhang et al., 2017).

Importantly it is seen that the gAMP treatment (“Guided”) is much more closely related to a simple probiotic treatment (“Empty”) than is the AMP treatment (“Unguided”) or mice given only a mock inoculation with buffer (“Null”). This means that treatment with probiotic expressing gAMP is much more like a normal probiotic treatment.

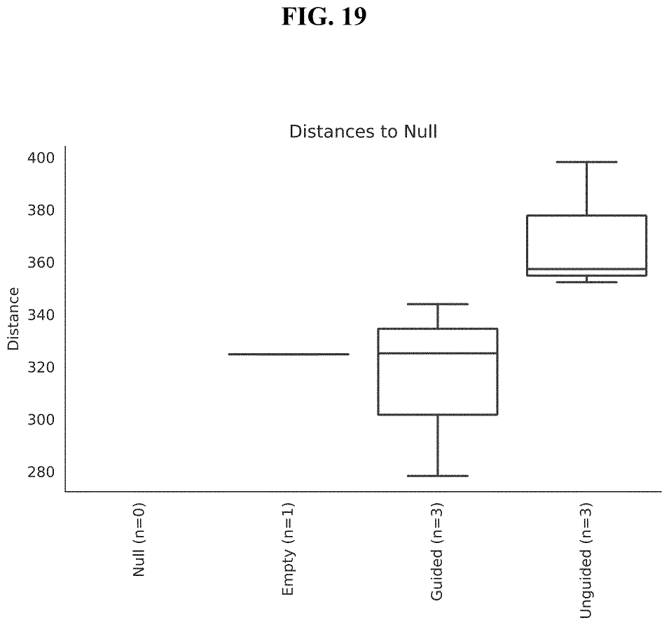

In FIG. 19, the same index is used, but with a comparison to the “Null” (mock inoculated) treatment. Again, the species assemblage found in the probiotic/gAMP (“Guided”) treatment is more closely related to the mock inoculated stomach microbial assemblage, as is the empty vector control. The “Unguided” (probiotic/AMP) assemblage is again more distantly related.

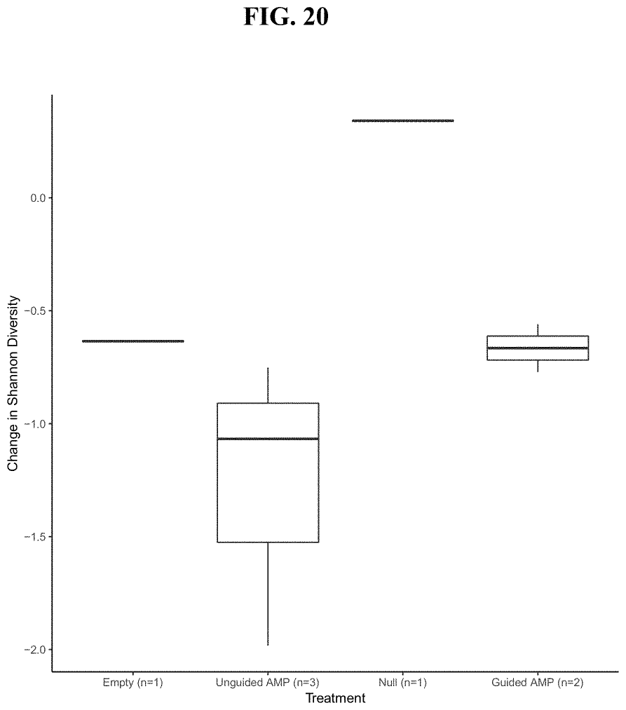

FIG. 20 measures the differences seen in species assemblages from the same treatment but at different time points. The index used is Shannon's entropy and it is reported in the y-axis. A more negative value (lower on the y-axis) indicates more change in the population over the 5 days since the inoculation of the mice on Day 0. It can be seen that the probiotic AMP (“Unguided”) treatment led to the greatest populational change over the 5 days. In contrast, the negative controls (“Empty” and “Null”) and the probiotic/gAMP (“Guided”) treatments led to only modest populational change. Error bars represent 95% confidence limits for all three figures.

### REFERENCES

The following patents and publications are hereby incorporated by reference.
WO 2017/009373
WO 2019/055781
U.S. Pat. No. 9,925,223
AU Patent No. 2016/204543
US Patent App. Pub. No. US 2013/0259834
US Patent App. Pub. No. US 2008/0044481
U.S. Patent App. Pub. No. US 2014/0066363
Baktash A, Terveer E M, Zwittink R D, et al. Mechanistic insights in the success of fecal microbiota transplants for the treatment of Clostridium difficile infections. Front Microbiol. 2018; 9:1242. doi:10.3389/fmicb.2018.01242
Börner R A, Kandasamy V, Axelsen A M, Nielsen A T, Bosma E F. Genome editing of lactic acid bacteria: opportunities for food, feed, pharma and biotecH. FEMS Microbiol Lett. 2019; 366(1):fny291. doi:10.1093/femsle/fny291
Borrero J, Chen Y, Dunny G M, Kaznessis Y N. Modified lactic acid bacteria detect and inhibit multiresistant enterococci. ACS Synth Biol. 2015; 4(3):299-306. doi:10.1021/sb500090b
Choudhury A, Islam S M A, Ghidey M R, Kearney C M. Repurposing a drug targeting peptide for targeting antimicrobial peptides against Staphylococcus. Biotechnol Lett. 2020; 42(2):287-294. doi:10.1007/s10529-019-02779-y
Deng T, Ge H, He H, et al. The heterologous expression strategies of antimicrobial peptides in microbial systems. Protein Expr Purif. 2017; 140:52-59. doi:10.1016/j.pep.2017.08.003
Ilan Y. Why targeting the microbiome is not so successful: can randomness overcome the adaptation that occurs following gut manipulation?. Clin Exp Gastroenterol. 2019; 12:209-217. Published 2019 May 8. doi:10.2147/CEG.S203823
Dowah A S A, Clokie M R J (2018) Review of the nature, diversity and structure of bacteriophage receptor binding proteins that target Gram-positive bacteria. Biophys Rev 10:535-542. doi: 10.1007/s12551-017-0382-3
Eckert R, He J, Yarbrough D K, et al (2006) Targeted killing of Streptococcus mutans by a pheromone-guided “smart” antimicrobial peptide. Antimicrob Agents Chemother 50:3651-3657. doi: 10.1128/AAC.00622-06
Eckert R, Sullivan R, Shi W (2012) Targeted Antimicrobial Treatment to Re-establish 194 a Healthy Microbial Flora for Long-term Protection. Adv Dent Res 24:94-97. doi: 10.1177/0022034512453725
Evans B C, Nelson C E, Yu S S, et al (2013) Ex vivo red blood cell hemolysis assay for the evaluation of pH-responsive endosomolytic agents for cytosolic delivery of biomacromolecular drugs. J Vis Exp e50166. doi:198 10.3791/50166
Everhart J E, Kruszon-Moran D, Perez-Perez G I, Tralka T S, McQuillan G. Seroprevalence and ethnic differences in Helicobacter pylori infection among adults in the United States. J Infect Dis. 2000; 181(4):1359-1363. doi:10.1086/315384
Fitchen N, Letley D P, O'Shea P, Atherton J C, Williams P, Hardie K R. All subtypes of the cytotoxin VacA adsorb to the surface of Helicobacter pylori post-secretion. J Med Microbiol. 2005; 54(Pt 7):621-630. doi:10.1099/jmm.0.45946-0
Forkus B, Ritter S, Vlysidis M, Geldart K, Kaznessis Y N. Antimicrobial Probiotics Reduce Salmonella enterica in Turkey Gastrointestinal Tracts. Sci Rep. 2017; 7:40695. Published 2017 Jan. 17. doi:10.1038/srep40695
Geldart K, Forkus B, McChesney E, McCue M, Kaznessis Y N. pMPES: A Modular Peptide Expression System for the Delivery of Antimicrobial Peptides to the Site of Gastrointestinal Infections Using Probiotics. Pharmaceuticals (Basel). 2016; 9(4):60. Published 2016 Oct. 5. doi:10.3390/ph9040060
Geldart K G, Kommineni S, Forbes M, et al. Engineered E. coli Nissle 1917 for the reduction of vancomycin-resistant Enterococcus in the intestinal tract. Bioeng Transl Med.
2018; 3(3):197-208. Published 2018 Sep. 8. doi:10.1002/btm2.10107 Ghidey M, Islam S M A, Pruett G, Kearney C M. Making plants into cost-effective bioreactors for highly active antimicrobial peptides. N Biotechnol. 2020; 56:63-70. doi:10.1016/j.nbt.2019.12.001
Granland C M, Scott N M, Lauzon-Joset J F, et al. Nasal Delivery of a Commensal Pasteurellaceae Species Inhibits Nontypeable Haemophilus influenzae Colonization and Delays Onset of Otitis Media in Mice. Infect Immun. 2020; 88(4):e00685-19. Published 2020 Mar. 23. doi:10.1128/IAI.00685-19
He B, Hoang T K, Wang T, et al. Resetting microbiota by Lactobacillus reuteri inhibits T reg deficiency-induced autoimmunity via adenosine A2A receptors. J Exp Med. 2017; 214(1):107-123.doi:10.1084/jem.20160961
Hooi J K Y, Lai W Y, Ng W K, et al. Global Prevalence of Helicobacter pylori Infection: Systematic Review and Meta-Analysis. Gastroenterology. 2017; 153(2):420-429. doi:10.1053/j.gastro.2017.04.022
Islam S M A, Kearney C M, Baker E J (2017) CSPred: A machine-learning-based compound model to identify the functional activities of biologically-stable toxins. In: 2017 IEEE International Conference on Bioinformatics and Biomedicine (BIBM). pp 2254-2255
Islam S M A, Sajed T, Kearney C M, Baker E J (2015) PredSTP: a highly accurate SVM based model to predict sequential cystine stabilized peptides. BMC Bioinformatics 16:210. doi:10.1186/s12859-015-0633-x
Keeney K M, Yurist-Doutsch S, Arrieta M C, Finlay B B. Effects of antibiotics on human microbiota and subsequent disease. Annu Rev Microbiol. 2014; 68:217-235. doi:10.1146/annurev-micro-091313-103456
Lange K, Buerger M, Stallmach A, Bruns T. Effects of Antibiotics on Gut Microbiota. Dig Dis. 2016; 34(3):260-268. doi:10.1159/000443360
Li C, Blencke H-M, Paulsen V, et al (2010) Powerful workhorses for antimicrobial peptide expression and characterization. Bioeng Bugs 1:217-220. doi: 10.4161/bbug.1.3.11721
Li, T., Qin, Y., Sham, P. et al. Alterations in Gastric Microbiota After H. pylori Eradication and in Different Histological Stages of Gastric Carcinogenesis. Sci Rep 7, 44935 (2017). doi.org/10.1038/srep44935
Li Z, Wang X, Wang X, et al (2017) Research advances on plectasin and its derivatives as new potential antimicrobial candidates. Process Biochemistry 56:62-70. doi: 10.1016/j.procbio.2017.02.006
Liou J M, Lee Y C, El-Omar E M, Wu M S. Efficacy and Long-Term Safety of H. pylori Eradication for Gastric Cancer Prevention. Cancers (Basel). 2019; 11(5):593. doi:10.3390/cancers11050593
Mao R, Teng D, Wang X, et al (2013) Design, expression, and characterization of a novel targeted plectasin against methicillin-resistant Staphylococcus aureus. Appl Microbiol Biotechnol 97:3991-4002. doi: 10.1007/s00253-012-210 4508-z
Monnet V, Juillard V, Gardan R (2016) Peptide conversations in Gram-positive bacteria. Crit Rev Microbiol 42:339-351. doi: 10.3109/1040841X.2014.948804
Mu Q, Tavella V J, Luo X M. Role of Lactobacillus reuteri in Human Health and Diseases. Front Microbiol. 2018; 9:757. Published 2018 Apr. 19. doi:10.3389/fmicb.2018.00757
Mygind P H, Fischer R L, Schnorr K M, et al (2005) Plectasin is a peptide antibiotic with therapeutic potential from a saprophytic fungus. Nature 437:975-980. doi: 10.1038/nature04051
Nguyen L T, Haney E F, Vogel H J (2011) The expanding scope of antimicrobial peptide structures and their modes 6 of action. Trends Biotechnol 29:464-472. doi: 10.1016/j.tibtech.2011.05.001
Nobrega F L, Vlot M, de Jonge P A, et al (2018) Targeting mechanisms of tailed bacteriophages. Nat Rev Microbiol 16:760-773. doi: 10.1038/s41579-018-0070-8
Oeemig J S, Lynggaard C, Knudsen D H, et al (2012) Eurocin, a new fungal defensin: structure, lipid binding, and its mode of action. J Biol Chem 287:42361-42372. doi: 10.1074/jbc.M112.382028
O'Toole G A (2011) Microtiter dish biofilm formation assay. J Vis Exp. doi: 10.3791/2437
Pan F, Zhang L, Li M, et al. Predominant gut Lactobacillus murinus strain mediates anti-inflammaging effects in calorie-restricted mice. Microbiome. 2018; 6(1):54. Published 2018 Mar. 21. doi:10.1186/s40168-018-0440-5
Parachin N S, Mulder K C, Viana A A B, et al (2012) Expression systems for heterologous production of antimicrobial peptides. Peptides 38:446-456. doi: 10.1016/j.peptides.2012.09.020
Peschel A, Sahl H-G (2006) The co-evolution of host cationic antimicrobial peptides and microbial resistance. Nat Rev Microbiol 4:529-536. doi: 10.1038/nrmicro1441
Saeidi N, Wong C K, Lo T M, et al. Engineering microbes to sense and eradicate Pseudomonas aeruginosa, a human pathogen. Mol Syst Biol. 2011; 7:521. Published 2011 Aug. 16. doi:10.1038/msb.2011.55
Satoh K, Hirayama T, Takano K, Suzuki-Inoue K, Sato T, Ohta M, Nakagomi J, Ozaki Y (2013) VacA, the vacuolating cytotoxin of Helicobacter pylori, binds to multimerin 1 on human platelets. Thrombosis J 11:23. doi: 10.1186/1477-9560-11-23
Thung I, Aramin H, Vavinskaya V, et al. Review article: the global emergence of Helicobacter pylori antibiotic resistance. Aliment Pharmacol Ther. 2016; 43(4):514-533. doi:10.1111/apt.13497
Vilander A C, Dean G A. Adjuvant Strategies for Lactic Acid Bacterial Mucosal Vaccines. Vaccines (Basel). 2019; 7(4):150. Published 2019 Oct. 16. doi:10.3390/vaccines7040150
Wang T, Teng K, Liu G, et al. Lactobacillus reuteri HCM2 protects mice against Enterotoxigenic Escherichia coli through modulation of gut microbiota. Sci Rep. 2018; 8(1):17485. Published 2018 Nov. 30. doi:10.1038/s41598-018-35702-y
Weber-Da̧browska B, Jończyk-Matysiak E, Zaczek M, et al (2016) Bacteriophage Procurement for Therapeutic Purposes. Front Microbiol 7:1177-1177. doi: 10.3389/fmicb.2016.01177
Worthington R J, Melander C (2013) Combination approaches to combat multidrug-resistant bacteria. Trends Biotechnol 31:177-184. doi: 10.1016/j.tibtech.2012.12.006
Wu C-H, Liu I-J, Lu R-M, Wu H-C (2016) Advancement and applications of peptide phage display technology in biomedical science. J Biomed Sci 23: doi: 10.1186/s12929-016-0223-x
Yacoby I, Shamis M, Bar H, et al (2006) Targeting Antibacterial Agents by Using Drug-Carrying Filamentous Bacteriophages. Antimicrobial Agents and Chemotherapy 50:2087-2097. doi: 10.1128/AAC.00169-06
Zhang L, Wu W K, Gallo R L, et al. Critical Role of Antimicrobial Peptide Cathelicidin for Controlling Helicobacter pylori Survival and Infection. J Immunol. 2016; 196(4):1799-1809. doi:10.4049/jimmunol.1500021
Zhang Y, Han S W, Cox L M, Li H. A multivariate distance-based analytic framework for microbial interdependence association test in longitudinal study. Genet Epidemiol. 2017; 41(8):769-778. doi:10.1002/gepi.22065

What is claimed is:

1. A probiotic for the prevention or treatment of a condition caused by a target bacterium living in the gastrointestinal tract of a subject, comprising:
a probiotic bacterium, wherein the probiotic bacterium has been transformed to comprise a DNA construct expressing a guided antimicrobial peptide, wherein the sequence coding for the guided antimicrobial peptide comprises the sequence coding for an antimicrobial peptide fused to the sequence coding for a guide peptide that binds to a protein of the target bacterium, wherein the guided antimicrobial peptide kills the target bacterium in the gastrointestinal tract of the subject, and wherein the guided antimicrobial peptide minimally disrupts other bacteria found in the gastrointestinal tract of the subject when compared to unguided antimicrobial peptides or antibiotics.

2. The probiotic of claim 1, wherein the probiotic bacterium comprises a lactic acid bacterium.

3. The probiotic of claim 2, wherein the lactic acid bacterium comprises a Lactococcus bacterium.

4. The probiotic of claim 3, wherein the Lactococcus bacterium comprises Lactococcus lactis.

5. The probiotic of claim 1, wherein the protein of the target bacterium is a virulence factor.

6. The probiotic of claim 5, wherein the virulence factor is the VacA peptide.

7. The probiotic of claim 1, wherein the antimicrobial peptide is laterosporulin, alyteserin, or cathelin-related anti-microbial peptide.

8. The probiotic of claim 1, wherein the target bacterium comprises H. pylori.

9. The probiotic of claim 1, wherein the guide peptide has a sequence comprising SEQ ID NO:5.

10. The probiotic of claim 1, wherein the antimicrobial peptide has a sequence comprising SEQ ID NO:6, SEQ ID NO:8, or SEQ ID NO:10.

11. The probiotic of claim 1, wherein the guided antimicrobial peptide has a sequence comprising SEQ ID NO:7, SEQ ID NO:9, or SEQ ID NO:11.

12. A probiotic composition for the prevention or treatment of a condition caused by a target bacterium living in the gastrointestinal tract of a subject, comprising:
the probiotic of claim 1; and
an acceptable excipient or carrier.

13. The probiotic composition of claim 1, wherein the probiotic bacterium is edible, and wherein the acceptable excipient or carrier is edible.

14. A method for preventing or treating a condition in a patient caused by a target bacterium found in the gastrointestinal tract of the subject, comprising:
administering a probiotic composition to the subject, wherein the probiotic composition comprises a probiotic bacterium and an acceptable excipient or carrier, wherein the probiotic bacterium has been transformed to comprise a DNA construct expressing a guided antimicrobial peptide, wherein the sequence coding for the guided antimicrobial peptide comprises the sequence coding for an antimicrobial peptide fused to the sequence coding for a guide peptide that binds to a protein of the target bacterium; and
allowing the guided antimicrobial peptide to kill the target bacterium in the gastrointestinal tract of the subject, and wherein the guided antimicrobial peptide minimally disrupts the other bacteria found in the gastrointestinal tract of the subject when compared to unguided antimicrobial peptides or antibiotics.

15. The method of claim 14, wherein the probiotic bacterium comprises a lactic acid bacterium.

16. The method of claim 15, wherein the lactic acid bacterium comprises a Lactococcus bacterium.

17. The method of claim 16, wherein the Lactococcus bacterium comprises Lactococcus lactis.

18. The method of claim 14, wherein the protein of the target bacterium is a virulence factor.

19. The method of claim 18, wherein the virulence factor is the VacA peptide.

20. The method of claim 14, wherein the antimicrobial peptide is laterosporulin, alyteserin, or cathelin-related anti-microbial peptide.

21. The method of claim 14, wherein the target bacterium comprises H. pylori.

22. The method of claim 14, wherein the guide peptide has a sequence comprising SEQ ID NO:5.

23. The method of claim 14, wherein the antimicrobial peptide has a sequence comprising SEQ ID NO:6, SEQ ID NO:8, or SEQ ID NO:10.

24. The method of claim 14, wherein the guided antimicrobial peptide has a sequence comprising SEQ ID NO:7, SEQ ID NO:9, or SEQ ID NO:11.

25. The method of claim 14, wherein the subject is an animal.

26. The method of claim 14, wherein the subject is a human.

27. The method of claim 14, wherein the probiotic bacterium is edible, and wherein the acceptable excipient or carrier is edible

28. The method of claim 14, wherein the probiotic composition is administered orally.

29. A probiotic for the prevention or treatment of a condition caused by Helicobacter pylori living in the gastrointestinal tract of a subject, comprising:
a Lactococcus lactis probiotic bacterium, wherein the Lactococcus lactis probiotic bacterium has been transformed to comprise a DNA construct expressing a guided antimicrobial peptide, wherein the the sequence coding for the guided antimicrobial peptide comprises the the sequence coding for an antimicrobial peptide fused to the sequence coding for a guide peptide that binds to the VacA peptide of H. pylori, wherein the guided antimicrobial peptide kills H. pylori in the gastrointestinal tract of the subject, and wherein the guided antimicrobial peptide minimally disrupts other bacteria found in the gastrointestinal tract of the subject when compared to unguided antimicrobial peptides or antibiotics.

30. The probiotic of claim 29 wherein the guide peptide is derived from multimerin-1 sequence.

31. The probiotic of claim 29, wherein the antimicrobial peptide is laterosporulin, alyteserin, or cathelin-related anti-microbial peptide.
